(19) United States
(12) Patent Application Publication
(10) Pub. No.: US 2023/0181543 A1
(43) Pub. Date: Jun. 15, 2023

(54) TREATMENT OF CARM1-OVEREXPRESSING AND/OR ARID1A MUTANT CANCERS WITH IRE-1/XBP-1 INHIBITORS

(71) Applicant: The Wistar Institute of Anatomy and Biology, Philadelphia, PA (US)

(72) Inventors: Rugang ZHANG, Philadelphia, PA (US); Joseph ZUNDELL, Philadelphia, PA (US); Jianhuang LIN, Philadelphia, PA (US)

(73) Assignee: The Wistar Institute of Anatomy and Biology, Philadelphia, PA (US)

(21) Appl. No.: 17/905,143

(22) PCT Filed: Feb. 26, 2021
(86) PCT No.: PCT/US2021/019856

§ 371 Date: Aug. 26, 2022

Related U.S. Application Data
(60) Provisional application No. 62/983,240, filed on Feb. 28, 2020.

(51) Int. Cl. A61K 31/436; A61P 35/00; A61K 45/06; C12Q 1/6886

(52) U.S. Cl. CPC A61K 31/436; A61P 35/00; A61K 45/06; C12Q 1/6886; C12Q 2600/106; C12Q 2600/156; C12Q 2600/158

(57) ABSTRACT

The present disclosure is directed to the use of IRE-1 and/or XBP-1 inhibitors to treat cancers having mutations in ARID 1 A over overexpressing CARM1.

### PRIORITY CLAIM

This application claims benefit of priority to U.S. Provisional Application Ser. No. 62/983,240, filed Feb. 28, 2020, the entire contents of which are hereby incorporated by reference.

### FEDERAL FUNDING STATEMENT

This invention was made with government support under Grant Nos. RO1CA163377, R01CA202919 and P30 CA010815-50 awarded by the National Institutes of Health. The government has certain rights in the invention

### BACKGROUND

### 1. Field

The present disclosure relates generally to the fields of medicine, oncology and genetics. More particularly, it provides methods of treating cancers with aberrations in CARM1 and/or ARID1A expression or structure.

### 2. Related Art

Epithelial ovarian cancer (EOC) remains the most lethal gynecological malignancy in the United States. High grade serous ovarian carcinoma (HGSOC) is the most common subtype (>70% of EOC cases) and accounts for the majority of EOC-associated mortalities. The survival rates of EOC patients still are remaining low due to the lack of therapeutic options. Notably, the treatment for homologous recombination proficient patients, such as BRCA wild-type patients, remains a problem due to its resistance to PARP inhibitor, the only approved targeted therapy for EOC.

Coactivator-associated arginine methyltransferase 1 (CARM1) is amplified or overexpressed in over 20% of HGSOC patients, and its high expression is associated with poor survival in HGSOC patients. Notably, CARM1 amplification/overexpression is typically mutually exclusive with homologous recombination deficiency such as that caused by BRCA1/2 mutations.

ARID1A is a DNA binding subunit of the SWI/SNF complex that is mutated in over 50% of ovarian clear cell carcinoma (OCCC) cases, which results in its loss of expression in over 90% of ARID1A-mutated OCCC cases. As with HGSOC, there is an urgent need for effective treatment approaches for ARID1A-mutated OCCCs since OCCCs are generally refractory to standard agents used to treat ovarian cancer and, when diagnosed in advanced stages, OCCCs carry the worst prognosis of all ovarian cancer subtypes.

Thus, there is a need to develop new therapeutic strategies for HGSOC and OCCC based on CARM1 and ARID1A status, respectively.

### SUMMARY

Thus, in accordance with the present disclosure, there is provided a method of treating a subject having a cancer that exhibits a mutation in ARID1A and/or overexpresses CARM1 as compared to a similar non-cancerous cell comprising administering to said subject an inhibitor of IRE-1/XBP-1. The inhibitor may be an IRE-1 selective inhibitor, an XBP-1 selective inhibitor, or an inhibitor of both IRE-1 and XBP-1. The method may further comprise treating said subject with a second cancer therapy, such as chemotherapy, radiotherapy, immunotherapy (e.g., checkpoint inhibitor), hormonal therapy, toxin therapy or surgery, either sequential or at the same time as said IRE-1/XBP-1 inhibitor. The immunotherapy may be a checkpoint inhibitor therapy.

The cancer may overexpress CARM1 as compared to a similar non-cancerous cell and/or may exhibit a mutation in ARID1A. The cancer may be an ovarian clear cell carcinoma cell or a high grade serous ovarian carcinoma cancer cell. The method may further comprise determining, prior to treating, that said subject carries an ARID1A-mutated cancer cell and/or a CARM1 overexpressing cancer cell. Determining may comprises (a) obtaining a sample from said subject that contains protein and/or nucleic acids; and (b1) determining mutation status of an ARID1A protein or nucleic acid encoding ARID1A in said sample, or (b2) determining the expression level of CARM1 in said sample. Determining may comprise a nucleic acid-based assay or a protein-based assay. The sample may be a fluid sample, such as blood, serum plasma, sputum, saliva, urine or nipple aspirate. The sample may be a tissue sample, such as a cancer tissue sample, such as a tumor biopsy.

The subject may be a human subject, such as a pediatric human subject, or a non-human primate. The subject may have previously been diagnosed with cancer, such as an OCCC cancer cell or an HGSOC cancer. The cancer may be recurrent, primary, metastatic or multi-drug resistant. The IRE-1/XBP-1 inhibitor may be administered more than once, such as daily, every other day, weekly, monthly and/or on a chronic basis. The method may comprise administering both and IRE-1 inhibitor and an EZH2 inhibitor, either sequentially or at the same time.

Also provided is a method of determining whether a subject having cancer should be treated with an inhibitor of IRE-1/XBP-1, wherein determining comprises (a) determining mutation status of an ARID1A protein or nucleic acid encoding ARID1A in a sample from said subject, and/or (b) determining the expression level of CARM1 in a sample from said subject. The cancer may be an ovarian clear cell carcinoma cell or a high grade serous ovarian carcinoma cancer cell, and/or is said cancer is recurrent, primary, metastatic or multi-drug resistant. Determining may comprise a nucleic acid-based assay or a protein-based assay. The sample may be a fluid sample, such as blood, serum plasma, sputum, saliva, urine or nipple aspirate, or may be a tissue sample, such as a cancer tissue sample, such as a tumor biopsy. The subject may be a human subject, such as a pediatric human subject, or may be a non-human primate. The subject may have previously been diagnosed with cancer, such as an OCCC cancer cell or an HGSOC cancer.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-B. CARM1 overexpression is associated with poor survival. (FIG. 1A) CARM1 amplification is mutually exclusive with BRCA1/2 mutations. (FIG. 1B) Overall survival of EOC patients with high or low CARM1 expression in an EOC microarray dataset.

FIGS. 2A-C. CARM1 expression is correlated with ER stress response/UPR signatures. (FIG. 2A) Experimental strategy used to establish CARM1's role in ER stress response/UPR. (FIG. 2B) Schematics of Unfolded Protein Response pathways. (FIG. 2C) CARM1 regulates the expression and activation of XBP-1.

FIGS. 3A-D. CARM1 expression sensitizes HGSOC cells to inhibition of IRE-1-XBP-1 pathway. (FIG. 3A) B-I09 (20 μM, 48 h) inhibits the activation of XBP-1. (FIG. 3B) Dose response curve of B-I09 in wild-type and CARM1 knockout A1847 cells. (FIG. 3C) The expression of CARM1 in different HGSOC cell lines. (FIG. 3D) The IC₅₀ of B-I09 in CARM1-low and high HGSOC cells.

FIGS. 4A-C. Inhibition of IRE-1-XBP-1 induces apoptosis in CARM1-high cells and it is dependent on unsaturated fatty acids synthesis. (FIG. 4A) Western blotting of apoptotic markers in A1847 cells with or without CARM1 knockout treated with or without B-I09. (FIGS. 4B-C) Schematics (FIG. 4B) and dose response curves (FIG. 4C) of A1847 cells with or without CARM1 knockout treated with B-I09 and rescued with BSA or oleic acid (OA).

FIG. 5. XBP-1 mRNA expression is significantly enhanced in ARID1A-mutant endometrial tumor samples. TCGA analysis of XBP-1 expression in endometrial tumor samples with wild-type ARID/A status compared to mutant ARID/A status.

FIGS. 6A-C. ARID1A interacts with the XBP-1 promoter and regulates its expression. (FIG. 6A) ChIP-seq showing ARID1A interaction with the XBP-1 promoter (FIG. 6B) anti-FLAG ChIP against endogenously expressed FLAG—ARID1A validating ARID1A interaction with the XBP-1 promoter (FIG. 6C) Immunoblot for RMG1 wild-type versus ARID1A knock-out (KO) cells analyzing XBP-1 protein expression after MG132 (20 PM), and tunicamycin (5 μg/mL) treatment.

FIGS. 7A-B. ARID1A-mutant cells are selectively sensitive to B-I09 inhibition. (FIG. 7A) IC₅₀ of B-I09 in cells possessing wild-type ARID1A status compared to mutant ARID1A status. Data from 3 replicates. (FIG. 7B) RMG1 and TOV21G cells were treated with B-I09 for 24 hours to show ARID1A mutant cell selectivity to ER stress response inhibition.

FIG. 8. ATF6 interacts with the XBP-1 promoter more in ARID1A-deficient cells. Anti-ATF6 ChIP in RMG1 wild-type parental control and CRISPR-Cas9 KO ARID1A OCCC cells. *p<0.05.

FIGS. 9A-B. B-I09 reduces tumor growth and enhances mouse survival in genetic OCCC mouse model. (FIG. 9A) Arid1a^{flox/flox};(Gt)Rosa26Pik3ca*^{H1047R} OCCC mice were injected with 50 mg/Kg B-I09 for 3 weeks (5 days on/2 off) and tumor weight was determined to show B-I09 efficacy in treating ARID1A mutant OCCC (5 mice per group). (FIG. 9B) Kaplan-Meier analysis from the OCCC model revealed greater survival after B-I09 treatment in tumor bearing mice (5 mice per group).

FIGS. 10A-D. Patient-derived xenograft OCCC model. Picture of female mouse ovary. (FIG. 10A), fallopian tube (FT) (FIG. 10B), and a patient-derived grafted tumor (FIG. 10C). (FIG. 10D) Reproductive tract of a tumor-bearing mouse. (Note: tumor cells were only engrafted into one ovary/bursa sac.)

FIG. 11. Loss of ARID1A promotes sensitivity to cisplatin and B-I09 combination. IC₅₀ of cisplatin in RMG1 wild-type compared to RMG1 ARID1A knock-out cells in combination with B-I09. Data from 3 replicates.

FIG. 12. Schematic of the overall research strategy hypotheses. ARID1A deficiency and platinum-based therapies cause ER stress. I hypothesize that ARID1A suppresses ER stress responses to maintain intracellular homeostasis. Loss of ARID1A promotes transcription of genes, such as XBP-1, by ATF6 to promote ovarian cancer cell survival. Targeting the ER stress response through B-I09 (IRE-1 RNase inhibitor) induces apoptosis and reduces tumor cell growth in OCCC.

FIGS. 13A-F. Identify CARM1 as a cofactor of XBP-1. (FIG. 13A) Expression of CARM1 and a loading control p-actin in CARM1-high parental and CRISPR-mediated CARM1 knockout (KO) A1847 cells. (FIG. 13B) Heatmap clustering of CUT$RUN seq profiles of CARM1 around TSSs in parental and CARM1 KO A1847 cells. CARM1 binding peaks in cluster 1 were used to identify CARM1 binding motif by MEME-ChIP and over-represented transcription factor (TF) binding motif by PscanChIP. XBP-1 was identified as an over-represented transcription factor in CARM1 binding peaks. (FIG. 13C) Tunicamycin was used to induced ER stress and spliced XBP-1 (XBP-1s) expression. XBP-1 protein levels was examined by western blotting. (FIG. 13D) Heatmap clustering of CUT$RUN seq profiles of XBP-1 and CARM1 in cells treated with or without tunicamycin. Cluster 1 showed the co-localization of XBP-1 and CARM1. (FIG. 13E) Average profiles of CUT&RUN seq signal for XBP-1 and CARM1 in cluster 1. (FIG. 13F) 1575 peaks from cluster1 were annotated to 1056 genes and gene ontology (GO) analysis showed main functional enrichment in ER related pathways.

FIGS. 14A-G. CARM1 regulates XBP-1-dependent transcription. (FIG. 14A) GSEA analysis of Unfolded Protein Response (UPR) gene signature in CARM1 wildtype and knockout RNA-seq. UPR gene signature was enriched in wildtype cells. (FIG. 14B) Heatmap showing the expression fold changes of UPR signature. Majority of the genes were downregulated in CARM1 knockout cells, and most of them are XBP-1 target genes (highlighted in red). (FIG. 14C) Positive gene expression correlation between CARM1 and UPR signature in TCGA-OV dataset (right panel) and CCLE dataset (left panel). (FIG. 14D) RT-qPCR results showing the expression of XBP-1 target genes in wildtype and CARM1 knockout A1847 and PEO4 cells. (FIG. 14E) Western blotting for validation of CARM1 knockout in PEO4 cells. (FIG. 14F) Reporter assay for XBP-1 binding motif, unfolded protein response element (UPRE). CARM1 knockout decreased XBP-1-dependent luciferase activity. (FIG. 14G) The expression of XBP-1 target genes HSPA5, HSPA9 and DNAJB9 upon tunicamycin treatment.

FIGS. 15A-F. IRE-1a-XBP-1 pathway is required for CARM1-expressing OvCa. (FIG. 15A) Colony formation assays of wildtype and CARM1 knockout A 1847 and PEO4 cells treated with IRE-1α inhibitor B-I09 or 4μ8c. B, Survival curves and IC₅₀ for the colony formation assays in panel A. Wild-type cells are significantly more sensitive to IRE-1α inhibitors. (FIG. 15C) Western blotting of apoptotic markers cleaved PARP and cleaved Lamin A and loading control β-actin. Treatment of B-I09 specifically induced apoptosis in wildtype CARM1-high cells. (FIG. 15D) The CARM1 levels in a panel of High Grade Serous Ovarian Cancer (HGSOC) cell lines were examined by western blotting. CARM1 is highly expressed in PEO4, OSAHO and A1847 cell lines. (FIG. 15E) Survival curves of colony formation assays of the panel of HGSOC cell lines treated with B-I09. (FIG. 15F) Comparison of IC₅₀ of CARM1-high and CARM1-low HGSOC cell lines under treatment of B-I09. CARM1-high cells showed dramatic lower IC₅₀.

FIGS. 16A-F. Interaction between CARM1 and XBP-1. (FIG. 16A) Immunoprecipitation assay with anti-CARM1 antibody to detect the interaction between CARM1 and XBP-1 in cells treated with or without tunicamycin. (FIG. 16B) Interaction between CARM1 and XBP-1 was further confirmed by GST-pulldown assay using purified GST-CARM1 and negative control GST. CARM1 but not GST interacts with XBP-1 in tunicamycin treated cell extracts. (FIG. 16C) Diagram of truncation mutants of CARM1. (FIG. 16D) GST-pull down assay using GST tagged CARM1 truncation mutants for domain mapping of CARM1 interaction domain. XBP-1 interacts with catalytic domain of CARM1. (FIG. 16E) In situ interaction between CARM1 and XBP-1 was examined by Proximity Ligation Assay (PLA). (FIG. 16F) Quantification of PLA signal showed the in situ interaction between CARM1 and XBP-1.

FIGS. 17A-C. CARM1 and XBP-1 cooperatively regulate ER stress response. (FIG. 17A) Representative CUT&RUN seq peaks of CARM1 and XBP-1 on indicated XBP-1 target gene loci in cell treated with or without tunicamycin. XBP-1, CARM1 and CARM1 substrate H3R17me2a co-localized at XBP-1 target gene promoters. (FIG. 17B) ChIP-qPCR analysis for the binding of XBP-1, CARM1 and H3R17me2a at indicated XBP-1 target gene promoters in cells treated with or without tunicamycin. The binding of XBP-1, CARM1 and H3R17me2a at XBP-1 target gene promoters was dramatically increased under ER stress. (FIG. 17C) ChIP-qPCR analysis for the binding of XBP-1, CARM1 and H3R17me2a at indicated XBP-1 target gene after knockdown of XBP-1.

FIGS. 18A-H. CARM1-high tumors are sensitive to IRE-1α-XBP-1 inhibition. (FIG. 18A) Hematoxylin and eosin (HE) staining, and immunohistochemical staining with anti-CARM1 antibody in CARM1-low and CARM1-high PDX. (FIG. 18B) Confirmation of CARM1 expression levels in CARM1-low and -high PDXs by western blotting. A1847 and OVCAR3 cell lysates were used as positive control for CARM1-high and CARM1-low expression, respectively. (FIGS. 18C-D) Schematic of experimental design for PDX mouse model was shown. Mice with indicated orthotopic CARM1-high and CARM1-low PDXs were randomized into two groups and treated with vehicle control or B-I09. Tumor weight was measured as a representation for tumor burden at the end of the treatment. (FIGS. 18E-F) Schematic of experimental design for orthotopic injection A1847 xenograft was shown. Mice with indicated orthotopic injection of wild-type or CARM1 knockout A1847 cells were randomized into two groups and treated with vehicle control or B-I09. Tumor weight was measured as a representation for tumor burden at the end of the treatment. (FIG. 18G) Kaplan-Meier survival curves for indicated xenograft groups. P value was calculated by log rank test. (FIG. 18H) Immunohistochemistry for apoptotic marker cleaved caspase 3 and proliferation marker Ki67, histological scores (H-score) were calculated for three separate fields from five tumors from five individual mice from each of the indicated groups.

FIGS. 19A-D. IRE-1a-XBP-1 inhibition synergizes with PD-1 immune therapy in CARM1-high ovarian cancer. (FIG. 19A) Western blotting of CARM1 and loading control □-actin in UPK10, A1847 and OVCAR3 cells. (FIG. 19B) Schematic of experimental design for orthotopic injection UPK10 xenograft was shown. B6 Mice with indicated orthotopic injection of UPK10 cells were randomized into four different treatment groups and treated with control vehicle, B-I09 (50 mg per kg, 5 weekdays per week by i.p.), PD-1 (10 mg per kg, twice per week) or a combination. (FIG. 19C) Tumor weight was measured as a representation for tumor burden at the end of the treatment. coefficient of drug interaction (CDI) was calculated. (FIG. 19D) Tumor infiltrated lymphocytes (TILs) was analyzed by flow cytometry. The percentage of CD4⁺ T cell, CD8⁺ T cell, B cell in CD69⁺ cell and macrophage were shown.

FIG. 20. ARID1A binds and regulates ER stress response genes.

FIG. 21. ARID1A mutant endometrial tumors exhibit high XBP-1 expression.

FIG. 22. ARID1A binds to the XBP-1 promoter.

FIG. 23. What is stress response?

FIG. 24. Targeting the ER stress response in cancer.

FIG. 25. Analyzing the ER stress response.

FIG. 26. XBP-1 expression is increased in ARID1A knock out cells.

FIG. 27. ARID1A knock out cells produce more XBP-1u during ER stress.

FIG. 28. What is known regarding the role of the mSWI/SNF complex.

FIG. 29. ARID1A guides SWI/SNF to transcriptionally regulate XBP-1 expression.

FIG. 30. ARID1A transcriptionally regulates XBP-1 expression during the ER stress response.

FIG. 31. ATF6 enhances XBP-1 gene expression when ARID1A expression is lost.

FIG. 32. ATF6 enhances XBP-1 gene expression when ARID1A expression is lost.

FIG. 33. ATF6 enhances XBP-1 gene expression when ARID1A expression is lost.

FIG. 34. ARID1A knock out cells exhibit an increase in ATF6 and XBP-1 target gene expression.

FIG. 35. ARID1A knock out cells exhibit an increase in ATF6 and XBP-1 target gene expression.

FIG. 36. Working mechanism for ER stress signaling.

FIG. 37. ARID1A mutated OCCC cells are sensitive to B-I09.

FIG. 38. ARID1A mutated OCCC cells are sensitive to B-I09.

FIG. 39. B-I09 selectively suppresses ARID1A mutant tumor formation.

FIG. 40. B-I09 selectively suppresses ARID1A mutant tumor formation.

FIG. 41. B-I09 suppresses tumor progression in an ARID1A/PIK3CA transgenic mouse model.

FIG. 42. XBP-1 knock out suppresses tumor progression in an ARID1A/PIK3CA transgenic mouse model.

FIG. 43. B-I09 suppresses tumor progression in an ARID1A/PIK3CA transgenic mouse model.

FIG. 44. CD8 depletion does not affect B-I09 treatment.

FIG. 45. B-I09 suppresses tumor growth in ARID1A-mutant OCCC PDX mice.

FIG. 46. Working mechanism for apoptosis driven by XBP-1 inhibition.

### DESCRIPTION OF ILLUSTRATIVE EMBODIMENTS

In this study, the inventors' preliminary data show that CARM1-high HGSOCs exhibit significantly higher levels of endoplasmic reticulum (ER) stress responses compared with CARM1-low or CARM1 knockout HGSOC cells. ER stress responses are frequently activated in human cancers and play pivotal roles in promoting tumor cell survival. Pharmacological inhibition of ER stress response pathway using the small molecule inhibitor B-I09 selectively induced apoptosis in CARM1-high but not CARM1-low or CARM1 knockout HGSOC cells. These results suggest that CARM1-high HGSOCs require ER stress response for their survival. The inventors' central hypothesis is that CARM1-high HGSOCs can be therapeutically targeted by the inhibition of the ER stress response.

In addition, the inventors performed ARID1A chromatin immunoprecipitation sequencing (ChIP-seq). They identified a direct interaction of ARID1A with the promoter of XBP-1, a key transcriptional regulator of endoplasmic reticulum (ER) stress. In addition, they showed that ARID1A-deficient OCCC cells enhance XBP-1 expression and are selectively sensitive to XBP-1 inhibition. However, the mechanistic details of this observed selectivity remain to be determined. In response to ER stress, IRE-1 splices XBP-1 mRNA into a functional transcription factor which enhances protein chaperone gene expression to restore cellular homeostasis. IRE-1-XBP-1 signaling is activated in many cancer types to promote cancer cell survival. Inhibition of this signaling pathway with B-I09 (an IRE-1 RNase inhibitor) has been effective in treating hematological malignancies.

These and other aspects of the disclosure are described in greater detail below.

### I. CARM1

CARM1 (coactivator-associated arginine methyltransferase 1), also known as PRMT4 (protein arginine N-methyltransferase 4), is an enzyme encoded by the CARM1 gene found in human beings, as well as many other mammals. It has a polypeptide (L) chain type that is 348 residues long and is made up of alpha helices and beta sheets. Its main function includes catalyzing the transfer of a methyl group from S-Adenosyl methionine to the side chain nitrogens of arginine residues within proteins to form methylated arginine derivatives and S-Adenosyl-L-homocysteine. CARM1 is a secondary coactivator through its association with p160 family (SRC-1, GRIP1, AIB) of coactivators. It is responsible for moving cells toward the inner cell mass in developing blastocysts. CARM1 plays an important role in androgen receptors and may play a role in prostate cancer progression.

CARM1 exerts both oncogenic and tumor-suppressive functions. In addition to its role in ovarians cancer, discussed above, in breast cancer CARM1 methylates chromatin remodeling factor BAF155 to enhance tumor progression and metastasis. In pancreatic cancer, CARM1 methylates and inhibits MDH1 by disrupting its dimerization, which represses mitochondria respiration and inhibits glutamine utilization. CARM1-mediated MDH1 methylation reduces cellular NADPH level and sensitizes cells to oxidative stress, thereby suppressing cell proliferation and colony formation.

### II. ARID1A

ARID1A (AT-rich interactive domain-containing protein 1A) is a protein that in humans is encoded by the ARID1A gene. The encoded protein is part of the large ATP-dependent chromatin remodelling complex SWI/SNF, which is required for transcriptional activation of genes normally repressed by chromatin. It possesses at least two conserved domains that could be important for its function. First, it has an ARID domain, which is a DNA-binding domain that can specifically bind an AT-rich DNA sequence known to be recognized by a SWI/SNF complex at the beta-globin locus. Second, the C-terminus of the protein can stimulate glucocorticoid receptor-dependent transcriptional activation. It is thought that the protein encoded by this gene confers specificity to the SW/SNF complex and may recruit the complex to its targets through either protein-DNA or protein-protein interactions. Two transcript variants encoding different isoforms have been found for this gene. This gene has been commonly found mutated in gastric cancers, ovarian clear cell carcinoma, and pancreatic cancer. In breast cancer distant metastases acquire inactivation mutations in ARID1A not seen in the primary tumor, and reduced ARID1A expression confers resistance to different drugs such as trastuzumab and mTOR inhibitors. These findings provide a rationale for why tumors accumulate ARID1A mutations.

### III. INHIBITORS

In accordance with the present disclosure, the inventors propose the use of IRE-1 and XBP-1 inhibitors to treat certain types of cancer, such as OCCC and HGSOC.

A. IRE-1 and Inhibitors Thereof

The serine/threonine-protein kinase/endoribonuclease inositol-requiring enzyme 1α (IRE-1α) is an enzyme that in humans is encoded by the ERN1 gene. The protein encoded by this gene is the ER to nucleus signalling 1 protein, a human homologue of the yeast IRE-1 gene product. This protein possesses intrinsic kinase activity and endoribonuclease activity and it is important in altering gene expression as a response to endoplasmic reticulum-based stress signals (mainly the unfolded protein response). Two alternatively spliced transcript variants encoding different isoforms have been found for this gene.

IRE-1α possesses two functional enzymatic domains, an endonuclease and a trans-autophosphorylation kinase domain. Upon activation, IRE-1α oligomerizes and carries out an unconventional RNA splicing activity, removing an intron from the X-box binding protein 1 (XBP-1) mRNA, and allowing it to become translated into a functional transcription factor, XBP-1s. XBP-1s upregulates ER chaperones and endoplasmic reticulum associated degradation (ERAD) genes that facilitate recovery from ER stress. ERN1 has been shown to interact with Heat shock protein 90 kDa alpha (cytosolic), member A1.

Two types of inhibitors exist targeting either the catalytic core of the RNase domain or the ATP-binding pocket of the kinase domain. RNase domain inhibitors include salicylaldehydes (3-methoxy-6-bromosalicylaldehyde, 4μ8C, MKC-3946, STF-083010, B-I09, toyocamycin, HNA and 3ETH. ATP-binding pocket inhibitors include sunitinib and APY29 inhibit the ATP-binding pocket but allosterically activate the IRE-1α RNase domain. Compound 3 prevents kinase activity, oligomerization and RNase activity. Various structures are shown below for convenience.

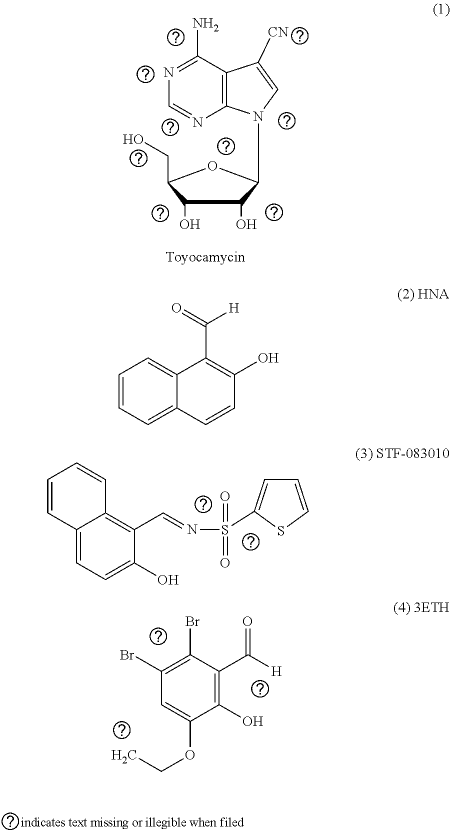

Other inhibitors include KIRA6, Kira8 HCl, KIRA-7, Sunitinib D10, MKC-8866, GSK2850163 and GSK2850163 HCl, AMG18 HCl, MKC-9989, OICR573, OICR464, D-F07 and 3,6-DMAD hydrochloride. Inhibitors for IRE-1/XBP-1 are disclosed in WO2019195135 and WO2014052669, hereby incorporated by reference.

B. XBP-1 and Inhibitors Thereof

X-box binding protein 1, also known as XBP-1, is a protein which in humans is encoded by the XBP-1 gene. The XBP-1 gene is located on chromosome 22 while a closely related pseudogene has been identified and localized to chromosome 5. The XBP-1 protein is a transcription factor that regulates the expression of genes important to the proper functioning of the immune system and in the cellular stress response. It contains a bZIP domain and was first identified by its ability to bind to the Xbox, a conserved transcriptional element in the promoter of the human leukocyte antigen (HLA) DR alpha. Abnormalities in XBP-1 lead to a heightened ER stress and subsequently causes a heightened susceptibility for inflammatory processes that may contribute to Alzheimer's disease. In the colon, XBP-1 anomalies have been linked to Crohn's disease.

The expression of XBP-1 is required for the transcription of a subset of class II major histocompatibility genes. Furthermore, XBP-1 heterodimerizes with other bZIP transcription factors such as c-fos. XBP-1 expression is controlled by the cytokine IL-4 and the antibody IGHM. XBP-1 in turn controls the expression of IL-6 which promotes plasma cell growth and of immunoglobulins in B lymphocytes.

XBP-1 is also essential for differentiation of plasma cells (a type of antibody secreting immune cell). This differentiation requires not only the expression of XBP-1 but the expression of the spliced isoform of XBP-1s. XBP-1 regulates plasma cell differentiation independent of its known functions in the endoplasmic reticulum stress response (see below). Without normal expression of XBP-1, two important plasma cell differentiation-related genes, IRF4 and Blimp1, are misregulated, and XBP-1-lacking plasma cells fail to colonize their long-lived niches in the bone marrow and to sustain antibody secretion. XBP-1 is also required for eosinophil differentiation. Eosinophils lacking XBP-1 exhibit defects in granule proteins.

XBP-1 acts to regulate endothelial cell proliferation through growth factor pathways, leading to angiogenesis. Additionally, XBP-1 protects endothelial cells from oxidative stress by interacting with HDAC3. It has also been identified as a cellular transcription factor that binds to an enhancer in the promoter of the Human T-lymphotropic virus 1. The generation of XBP-1s during plasma cell differentiation also seems to be the cue for Kaposi's sarcoma-associated herpesvirus and Epstein Barr virus reactivation from latency.

XBP-1 is part of the endoplasmic reticulum (ER) stress response, the unfolded protein response (UPR). Conditions that exceed capacity of the ER provoke ER stress and trigger the unfolded protein response (UPR). As a result, GRP78 is released from IRE-1 to support protein folding. IRE-1 oligomerises and activates its ribonuclease domain through auto (self) phosphorylation. Activated IRE-1 catalyses the excision of a 26-nucleotide unconventional intron from ubiquitously expressed XBP-1u mRNA, in a manner mechanistically similar to pre-tRNA splicing. Removal of this intron causes a frame shift in the XBP-1 coding sequence resulting in the translation of a 376 amino acid, 40 kDa, XBP-1s isoform rather than the 261 amino acid, 33 kDa, XBP-1u isoform. Moreover, the XBP-1u/XBP-1s ratio (XBP-1-unspliced/XBP-1-spliced ratio) correlates with the expression level of expressed proteins in order to adapt the folding capacity of the ER to the respective requirements.

In general, inhibition of XBP-1 is generally achieved by targeting IRE-1, which in turns inhibits XBP-1.

C. EZH2 and Inhibitors Thereof

Enhancer of zeste homolog 2 (EZH2) is a histone-lysine N-methyltransferase enzyme encoded by EZH2 gene, that participates in histone methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis.

EZH2 is the functional enzymatic component of the Polycomb Repressive Complex 2 (PRC2), which is responsible for healthy embryonic development through the epigenetic maintenance of genes responsible for regulating development and differentiation. EZH2 is responsible for the methylation activity of PRC2, and the complex also contains proteins required for optimal function (EED, SUZ12, JARID2, AEBP2, RbAp46/48, and PCL).

Mutation or over-expression of EZH2 has been linked to many forms of cancer. EZH2 inhibits genes responsible for suppressing tumor development and blocking EZH2 activity may slow tumor growth. EZH2 has been targeted for inhibition because it is upregulated in multiple cancers including, but not limited to, breast, prostate, melanoma, and bladder cancer. Mutations in the EZH2 gene are also associated with Weaver syndrome, a rare congenital disorder, and EZH2 is involved in causing neurodegenerative symptoms in the nervous system disorder, ataxia telangiectasia.

Developing an inhibitor of EZH2 and preventing unwanted histone methylation of tumor suppressor genes is a viable area of cancer research. EZH2 inhibitor development has focused on targeting the SET domain active site of the protein. Several inhibitors of EZH2 have been developed as of 2015, including 3-deazaneplanocin A (DZNep), EPZ005687, EI1, GSK126, and UNC1999. DZNep has potential antiviral and anti-cancer properties because it lowers EZH2 levels and induces apoptosis in breast and colon cancer cells. DZNep inhibits the hydrolysis of S-adenosyl-L-homocysteine (SAH), which is a product-based inhibitor of all protein methyltransferases, leading to increased cellular concentrations of SAH which in turn inhibits EZH2. However, DZNep is not specific to EZH2 and also inhibits other DNA methyltransferases. In 2012, a company called Epizyme revealed EPZ005687, an S-adenosylmethionine (SAM) competitive inhibitor that is more selective than DZNep; it has a 50-fold increase in selectivity for EZH2 compared to EZH1. The drug blocks EZH2 activity by binding to the SET domain active site of the enzyme. EPZ005687 can also inhibit the Y641 and A677 mutants of EZH2, which may be applicable for treating non-Hodgkin's lymphoma. In 2013, Epizyme began Phase I clinical trials with another EZH2 inhibitor, tazemetostat (EPZ-6438), for patients with B-cell lymphoma.

Sinefungin is another SAM-competitive inhibitor, however, like DZNep, it is not specific to EZH2. It works by binding in the cofactor binding pocket of DNA methyltransferases to block methyl transfer. EI1 is another inhibitor, developed by Novartis, that showed EZH2 inhibitory activity in lymphoma tumor cells, including cells with the Y641 mutation. The mechanism of this inhibitor also involves competing with the SAM cofactor for binding to EZH2. GSK126 is a potent, SAM-competitive EZH2 inhibitor developed by GlaxoSmithKline, that has 150-fold selectivity over EZH1 and a K, of 0.5-3 nM. UNC1999 was developed as an analogue of GSK126, and was the first orally bioavailable EZH2 inhibitor to show activity. However, it is less selective than its counterpart GSK126, and it binds to EZH1 as well, increasing the potential for off-target effects.

Combination therapies are being studied as possible treatments when primary treatments begin to fail. Etoposide, a topoisomerase inhibitor, when combined with an EZH2 inhibitor, becomes more effective for non-small cell lung cancers with BRG1 and EGFR mutations. However, EZH2 and lysine methylation can have tumor suppressing activity, for example in myelodysplastic syndrome, indicating that EZH2 inhibition may not be beneficial in all cases.

Treatment of CARM1-overexpressing cancers with EZH2 inhibitors is disclosed in WO2017192290, hereby incorporated by reference. Treatment of ARID1A mutated cancers with EZH2 inhibitors is disclosed in WO2018034801, hereby incorporated by reference.

### IV. TREATING CANCERS

A. Cancers

Cancer encompasses a group of diseases involving abnormal cell growth with the potential to invade or spread to other parts of the body. These contrast with benign tumors, which do not spread to other parts of the body. Possible signs and symptoms include a lump, abnormal bleeding, prolonged cough, unexplained weight loss and a change in bowel movements. While these symptoms may indicate cancer, they may have other causes. Over 100 types of cancers affect humans.

Cancer can spread from its original site by local spread, lymphatic spread to regional lymph nodes or by hematogenous spread via the blood to distant sites, known as metastasis. When cancer spreads by a hematogenous route, it usually spreads all over the body. The symptoms of metastatic cancers depend on the tumor location and can include enlarged lymph nodes (which can be felt or sometimes seen under the skin and are typically hard), enlarged liver or enlarged spleen, which can be felt in the abdomen, pain or fracture of affected bones and neurological symptoms.

Many treatment options for cancer exist. The primary ones include surgery, chemotherapy, radiation therapy, hormonal therapy, targeted therapy and palliative care. Which treatments are used depends on the type, location and grade of the cancer as well as the patient's health and personal wishes. The treatment intent may or may not be curative.

The therapeutic methods of the disclosure in general include administration of a therapeutically effective amount of compositions described herein to a subject in need thereof, including a mammal, particularly a human. Such treatment will be suitably administered to subjects, particularly humans, suffering from cancer or having a symptom thereof.

Cancers may be a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. More specifically, the tumors will have mutations in ARID1a and/or overexpress CARM1. Of particular interest are certain ovarian cancers of epithelial origin having the types of aberrations, as discussed below.

1. HGSOC

High-grade serous carcinoma (HGSC) is a type of tumor that arises from the serous epithelial layer in the abdominopelvic cavity and is mainly found in the ovary. HGSCs make up the majority of ovarian cancer cases (HGSOCs) and have the lowest survival rates. HGSC is distinct from low-grade serous carcinoma (LGSC) which arises from ovarian tissue, is less aggressive and is present in stage I ovarian cancer where tumors are localised to the ovary.

Although originally thought to arise from the squamous epithelial cell layer covering the ovary, HGSC is now thought to originate in the Fallopian tube epithelium. HGSC is much more invasive than LGSC with a higher fatality rate—although it is more sensitive to platinum-based chemotherapy, possibly due to its rapid growth rate. In rare cases, HGSCs can develop from LGSCs, but generally the two types arise independently of each other.

The ‘incessant ovulation’ theory is suggested by the strong correlation between the number of ovulatory cycles of an individual and their risk of ovarian cancer. This trend is reflected in the protective effects of pregnancy, parity and breastfeeding against ovarian cancer, and similar findings in epidemiological studies that have indicated a reduction of risk associated with use of oral contraceptive pills.

Ovulation is accepted as the cause of ovarian cortical inclusion cysts, the precursor lesions of serous carcinomas, and lower numbers of these cortical inclusion cysts are thought to be the mechanism by which reducing lifetime ovulations can lower the risk of developing HGSC. Conversely, a temporal association with menopausal hormone therapy and incidence of HGSC was found, and polycystic ovarian syndrome (PCOS) was shown to contribute to a doubling of the risk of ovarian cancer. Endometriosis can increase risk for other ovarian cancer subtypes but is not associated with HGSC.

More than 20% of ovarian cancer tumors have hereditary origin. The majority of these feature mutations in the tumor suppressor BRCA genes, which tend to give rise to HGSC. A mutation in BRCA1 or BRCA2 can confer a lifetime ovarian cancer risk of 40-50% and 10-20% respectively, with BRCA2 mutations strongly associated with better clinical outcomes.

A specific tumor protein 53 (TP53) expression pattern in the Fallopian tube epithelium—the ‘p53 signature’—is thought to be a precursor marker of HGSC. TP53^{-/-} mice (in which the TP53 gene has been deleted) do not develop ovarian carcinomas. However, TP53 mutations were found in 96% of HGSC cases. A local abnormal TP53 expression may thus be indicative of HGSC. In women, pelvic HGSC show either a complete absence of P53 expression, or overexpression, suggesting that any aberration of P53 leads to tumor development. Additionally, overexpression of TP53 is associated with better clinical outcome whereas an absence of the p53 protein is linked to an increased risk of HGSC tumor recurrence.

HGSC are further distinguished from LGSC by ‘type I/II’ ovarian tumor nomenclature; type I referring to tumor types (e.g., LGSCs) where precursor lesions within the ovary have been characterised, and type II referring to tumor types (e.g., HGSCs) without association of such lesions, tumors understood to develop de novo from the tubal and/or ovarian surface epithelium. This classification has more relevance to research rather than to clinical practice.

The serous membrane is a particular type of secretory epithelium which covers organs in body cavities and secretes serous fluid to reduce friction from muscle movement. Serous membrane lining the abdominopelvic cavity is called the peritoneum; that lining heart and mediastinum is the pericardium, and that lining the thoracic cavity and lungs is the pleura. Technically a ‘serous carcinoma’ can occur anywhere on these membranes, but high-grade serous carcinoma is generally limited to the peritoneal area.

While until recently HGSC was thought to arise from simple differentiation of cortical inclusion cysts (CICs) of ovarian surface epithelium (OSE), the cell origin of HGSC is now understood to be much more complex, with evidence for other sites of origin, both intra- and extra-ovarian, having come to light. The common Müllerian origin of the Fallopian tubes, uterus, cervix, and upper vagina has resulted in the proposal that peritoneal high-grade serous carcinoma is a spectrum of a single disease. The specific process by which a HGSC arises may be related to the BRCA mutation status of the individual, as well as the p53 mutation status.

Assuming a fimbrial origin, as observed in the majority of HGSC cases, the current understanding of HGSC genesis suggests a process by which STIC fimbrial cells implant into the ovary as cortical inclusion cysts through the ovulation rupture site. To account for instances where there is no STIC involvement, endosalpingiosis or de novo metaplasia of ovarian surface epithelium inclusions are also possible. A much rarer occurrence is the differentiation of HGSC from LGSC.

Diagnosis is initially through symptoms including persistent bloating, postmenopausal bleeding, and/or appetite loss. Transvaginal ultrasonography as well as cancer marker CA125 level analysis is often used to determine potential malignancy of suspect pelvic masses. Surgical staging is the procedure by which the abdominal cavity and lymph nodes are examined for malignant tissue, usually via laparoscopy. Tissue biopsies may be taken for further analysis. It is not until this histological analysis stage that actual diagnosis of HGSC can be made. If glands are seen to fuse with intricate, extensive papillae featuring epithelial tufting with solid nests surrounded by a space alongside irregular slit-like spaces, then serous carcinoma is suspected.

In terms of distinguishing between LGSC and HGSC, necrosis is common in HGSC and absent in LGSC, as are giant (multi- or mononucleated) tumor cells. Psammoma bodies are more frequent in low-grade serous carcinoma. TP53 expression is assessed for mutations, overexpression or absence—common features of high-grade serous carcinomas. LGSCs are generally limited to micropapillary growth patterns, whereas HGSCs can exhibit admixed patterns.

Distinction of HGSC from high-grade endometrioid carcinoma is not always possible. The progression of HGSC may also be determined from examining the cadherin expression profile.

As ovarian cancer is rarely symptomatic until an advanced stage, regular pre-emptive screening is a particularly important tool for avoiding the late stage at which most patients present. While a U.S. study found that transvaginal ultrasound and cancer marker CA125 screening did not reduce ovarian cancer mortality, a more recent U.K. study found that up to 20% of ovarian cancer deaths could be prevented through annual performance of these procedures.

In terms of treatment, cytoreductive “debulking” surgery may be performed prior to chemotherapy treatment in order to decrease the physical mass of the tumor and thus reduce the number of chemotherapy cycles needed. The typical advanced presentation as well as extra-ovarian spread seen in HGSC can require aggressive debulking procedures. In some cases, total abdominal hysterectomy will be performed, in other cases where the patient intends to bear children a salpingo-oophorectomy is performed instead.

Typical chemotherapy is six cycles of intraperitoneally-delivered platinum-base adjuvant chemotherapy with agents such as carboplatin. Measurements of blood CA125 levels are used to determine patient response to the treatment. Between 20% and 30% of patients relapse within six months of treatment. Poly ADP ribose polymerase (PARP) inhibitors are another possible treatment, with carriers of BRCA1/2 mutations being the most responsive.

2. OCCC

Ovarian clear cell carcinoma (OCCC) is one of several subtypes of ovarian carcinoma. The two types of ovarian carcinoma are epithelial and nonepithelial. Within these two categories, clear cell is a subtype of epithelial ovarian cancer. The other major subtypes within this group include high-grade serous, endometrioid, mucinous, and low-grade serous. The serous type is the most common form of epithelial ovarian tumors. Cord-stromal and germ cell belong to the nonepithelial category which are much less common. According to research, most ovarian cancers start at the epithelial layer which is the lining of the ovary. Within this epithelial group ovarian clear cell carcinoma makes up about 5-10%. Its incidence rate differs across various ethnic groups. Reports from the United States show that the highest rates are among Asians with 11.1% versus whites with 4.8% and blacks at 3.1%. These numbers are consistent with the finding that although clear cell carcinomas are rare in western countries, they are much more common in parts of Asia.

Ovarian clear cell carcinoma often occurs as a pelvic mass that rarely appears bilaterally. The cells usually contain glycogen with large clear cytoplasm. It is also associated with endometriosis, a disorder of abnormal tissue growth outside of the uterus. The tumor cells emerge in a stepwise manner from adenofibromas which are benign endometriotic cysts. They also hold molecular genetic mutations in both ARID1A and PIK3CA, similar to other epithelial ovarian cancers. Mutations in ARID1A commonly contain phosphatase and tensin homolog (PTEN) that are hypothesized to contribute to clear cell tumorigenesis. However, research also shows that inactivation of ARID1A alone does not lead to tumor initiation, but clear cell tumors rarely carry p53, BRCA1, or BRCA2 mutations. In addition, they also test negative for estrogen and progesterone receptors and Wilm tumor suppressor 1. Studies have also suggested that ovarian clear-cell carcinoma can occur with thromboembolic complications and hypercalcemia. Recurrence of tumor cells have been reported to involve lymph nodes and parenchymal organs.

A suggested mechanism for OCCC progression is the amplification and overexpression of CCNE1 which is thought to promote the tumors aggressive behavior. In addition, they also test negative for estrogen and progesterone receptors and Wilm tumor suppressor 1. The CCNE1 gene encodes for the cyclin E1 protein which accumulates at the G1-S phase transition point of the cell cycle. Detecting the cancerous tumor progression can be difficult for pathologists. While some tumors will appear in the ovary, others spread over the outer lining of the ovary and to other organs such as the uterus, fallopian tube, and lymph glands.

Ovarian clear cell tumors are frequently found at an early stage and therefore can be cured with surgery. Through clinical examination or preoperative imaging techniques, tumors have been reported to range from 3-20 cm. Most ovarian tumors are benign and rarely spread past the ovary. Therefore, surgical removal of the ovary or partial removal of the ovary is sufficient for treatment for malignant tumors. When diagnosed beyond FIGO (International Federation of Gynecology and Obstetrics) stage 1, patients usually have a poor prognosis. If the malignant tumors metastasize and spread throughout the body, then they could potentially be fatal. Ovarian clear cell tumors have been found to be resistant to conventional chemotherapy using platinum and taxane. Although the cause of this chemoresistance is unknown, there is research that provides partial explanation of this occurrence. For example, studies show that ovarian clear cell tumor cells proliferate at lower rates than serous adenocarcinomas which then could aid in a lower response from clear cell tumors to chemotherapies.

Given that treatment options are limited for clear cell ovarian cancer patients, researchers are studying biomarkers or specific pathways that could aid in developing future treatment. These patients are good candidates for targeted therapies since the standard does not adequately help their care. Some suggested therapeutic targets include the PI3K/AKT/mTOR, VEGF, Il-6/STAT3, MET, and HNF-1beta pathways.^{[8]} Better insight into genomic heterogeneity would also provide a personalized approach to identifying treatment targets for clear cell tumor patients that share similar phenotypes. Developing stronger options is also beneficial because ovarian cancer is the fifth leading cause of cancer deaths for women and is one of the most lethal gynecological cancers.

B. Diagnostic/Theranostic Methods

In one embodiment, the disclosure provides methods to assess the ARID1A mutational status and/or CARM1 expression of a cancer being treated. The method includes the step of determining whether a cancer patient's cancer has ARID1A mutations and/or overexpresses CARM1 prior to administering a therapeutic composition as described herein. The analysis is useful in predicting whether the subject will respond to a glutamate metabolism inhibitor—if so, then the glutamate inhibitor is administered, and if not, then another therapy is employed. The following exemplary techniques can be employed to examine the ARID1A mutational status and/or CARM1 expression.

1. Nucleic Acid-Based Detection Methods

Nucleic acid-based detection methods may be employed to identify cancers with mutant ARID1A. They may also be used in a quantitative fashion to examine the protein expression of CARM1. The following is a discussion of such methods, which are applicable to assessing mutations in ARID1A and CARM1 expression. In certain embodiments, the disclosure relates to methods of characterizing and treating cancer by detecting mutant ARID1A and/or detecting overexpression of CARM1. The methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice.

i. Hybridization

Methods looking at DNA or mRNA all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

ii. Nucleic Acid Amplification

Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best-known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously. The presence of many PCR primers in a single tube could cause many problems, such as the increased formation of misprimed PCR products and “primer dimers,” the amplification discrimination of longer DNA fragment and soon. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller and Wold (1989).

Another method for amplification is ligase chain reaction (“LCR”), disclosed in European Application No. 320 308, incorporated herein by reference in its entirety. U.S. Pat. 4,883,750 describes a method similar to LCR for binding probe pairs to a target sequence. A method based on PCR7 and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence-based amplification (NASBA) and 3SR (Kwoh el al., 1989; Gingeras el al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

iii. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook el al., 1989). Separated amplification products may be cut out and eluted from the gel for further manipulation. Using low melting point agarose gels, the separated band may be removed by heating the gel, followed by extraction of the nucleic acid.

Separation of nucleic acids may also be achieved by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

iv. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, Si nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

2. Protein-Based Detection Methods

i. Immunodetection

In still further embodiments, there are immunodetection methods for identifying and/or quantifying CARM1 overexpression. These methods may, in certain embodiments, be applied to the treatment of cancer, such as those discussed above.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of TSP1 antibodies also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample and contacting the sample with a first antibody in accordance with embodiments discussed herein, as the case may be, under conditions effective to allow the formation of immunocomplexes.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to CARM1 present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

ii. ELISAs

Immunoassays, in their most simple sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the TSP1 is added to the wells. After binding and washing to remove non-specifically bound immune complexes, the bound antigen may be detected. Detection may be achieved by the addition of another anti-CARM1 antibody that is linked to a detectable label. This type of ELISA is a simple “sandwich ELISA.” Detection may also be achieved by the addition of a second anti-CARM1 antibody, followed by the addition of a third antibody that has binding affinity for the second antibody, with the third antibody being linked to a detectable label.

In another exemplary ELISA, the samples suspected of containing the CARM1 are immobilized onto the well surface and then contacted with anti-CARM1 antibody. After binding and washing to remove non-specifically bound immune complexes, the bound anti-CARM1 antibodies are detected. Where the initial anti-CARM1 antibodies are linked to a detectable label, the immune complexes may be detected directly. Again, the immune complexes may be detected using a second antibody that has binding affinity for the first anti-CARM1 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

iii. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their nonspecific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

iv. Immunohistochemistry

The antibodies may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown el al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

v. Immunodetection Kits

In still further embodiments, there are immunodetection kits for use with the immunodetection methods described above. The immunodetection kits will thus comprise, in suitable container means, a first antibody that binds to TSP1 antigen, and optionally an immunodetection reagent.

In certain embodiments, the TSP1 antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with embodiments discussed herein.

The kits may further comprise a suitably aliquoted composition of the TSP1 antigen, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits will also include a means for containing the antibody, antigen, and any other reagent containers in close confinement for commercial sale. Such containers may include injection or blow-molded plastic containers into which the desired vials are retained.

vi. Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) while newer quantitative methods are being developed using matrix assisted laser desorption/ionization (MALDI) followed by time of flight (TOF) MS. In particular, mass spectrometry has been applied to samples to identify proteins targets therein.

ESI is a convenient ionization technique that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals and bioactive peptides. Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Protein quantification has been achieved by quantifying tryptic peptides. Complex mixtures such as crude extracts can be analyzed, but in some cases sample clean up is required.

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., 0, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to different size causing different velocity. Each ion mass will thus have a different flight-time to the detector.

One can either form positive ions or negative ions for analysis. Positive ions are made from regular direct photoionization, but negative ion formation requires a higher-powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectrum.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

Since its inception and commercial availability, the versatility of MALDI-TOF-MS has been demonstrated convincingly by its extensive use for qualitative analysis. For example, MALDI-TOF-MS has been employed for the characterization of synthetic polymers, peptide and protein analysis, DNA oligonucleotide sequencing, and the characterization of recombinant proteins. Recently, applications of MALDI-TOF-MS have been extended to include the direct analysis of biological tissues and single cell organisms with the aim of characterizing endogenous peptide and protein constituents.

The properties that make MALDI-TOF-MS a popular qualitative tool-its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use. These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products, whereas other studies have demonstrated the potential of MALDI-TOF-MS for the quantification of biologically relevant analytes such as neuropeptides, proteins, antibiotics, or various metabolites in biological tissue or fluid. In earlier work, it was shown that linear calibration curves could be generated by MALDI-TOF-MS provided that an appropriate internal standard was employed. This standard can “correct” for both sample-to-sample and shot-to-shot variability. Stable isotope labeled internal standards (isotopomers) give the best result.

With the marked improvement in resolution available on modern commercial instruments, primarily because of delayed extraction, the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated but are not used routinely.

C. Combination Therapies

It may also be useful to treat cancers using the methods and compositions of the present disclosure, but further empty at least one other therapy. The present therapy and the other therapy would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the antibody may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where the IRE-1 and/or XBP-1 inhibitor of the present disclosure is “A,” and the other therapy (e.g., EZH2) is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Some agents or therapies suitable for use in a combined therapy with agents according to the present disclosure against cancer are discussed below, although other combinations are contemplated. The following is a general discussion of cancer therapies that may be used combination with the compounds of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins, mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including cytokines such as IL-2, IL-4, IL-12, GM-CSF, γ-IFN, chemokines such as MIP-1, MCP-1, IL-8, and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg el al., 1988; 1989).

4. Surgery

Approximately 60%6 of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the reoccurrence of the tumor. Additionally, the compounds of the present disclosure can also be used in a neoadjuvant setting.

It also should be pointed out that any of the foregoing therapies may prove useful by themselves in treating cancer.

### V. EXAMPLES

The following examples are included to demonstrate preferred embodiments. It should be appreciated by those of skill in the art that the techniques disclosed in the examples which follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—CARM1

Epithelial ovarian cancer (EOC) remains the most lethal gynecological malignancy in the United States (Siegel et al., 2019). EOC is comprised of multiple separate diseases. High grade serous ovarian carcinoma (HGSOC) is the most common subtype (>70% of EOC cases) and accounts for the majority of EOC-associated mortalities. The standard chemotherapy drugs used to treat HGSOC is the combination of platinum and taxane (Pujade-Lauraine et al. 2014). However, the survival rates of patients remain low due to the development of chemotherapeutic resistance and lack of new therapeutic options. Therefore, there is an unmet need to develop new therapeutic options, such as targeted therapy to improve the survival for EOC patients. Analysis of HGSOC patients from The Cancer Genome Atlas (TCGA) has identified that Coactivator-associated arginine methyltransferase 1 (CARM1) is amplified and/or overexpressed in 20% of HGSOC patients (Karakashev et al., 2018; Cancer Genome ARS, 2011). CARM1-high tumors represent a genetically distinct subtype of HGSOC that is characterized by worse prognosis.

CARM1 is a type I protein arginine methyltransferase that asymmetrically dimethylates protein substrates on arginine residue. CARM1 has been proposed to function as an oncogene in different types of cancer. However, directly targeting CARM1 is not likely feasible because it is required for postnatal survival (Yadav et al., 2003). Notably, the roles of CARM1 in EOC remain elusive. The TCGA analysis revealed that CARM1 is amplified in ˜10% of HGSOC (FIG. 1A) and overexpressed in another ˜10% of HGSOC, which represents the highest rate among all cancer types. The high expression of CARM1 correlates with shorter overall survival (FIG. 1B). Moreover, CARM1 amplification is typically mutually exclusive with homologous recombination deficiency such as that caused by BRCA1/2 mutations (FIG. 1A). This suggests that patients with CARM1-high tumors cannot benefit from PARP inhibitors, the only targeted therapy for EOC that targets HR deficient HGSOCs under current clinical guidelines. Thus, there is an urgent need for developing therapeutic approaches against CARM1-high HGSOCs.

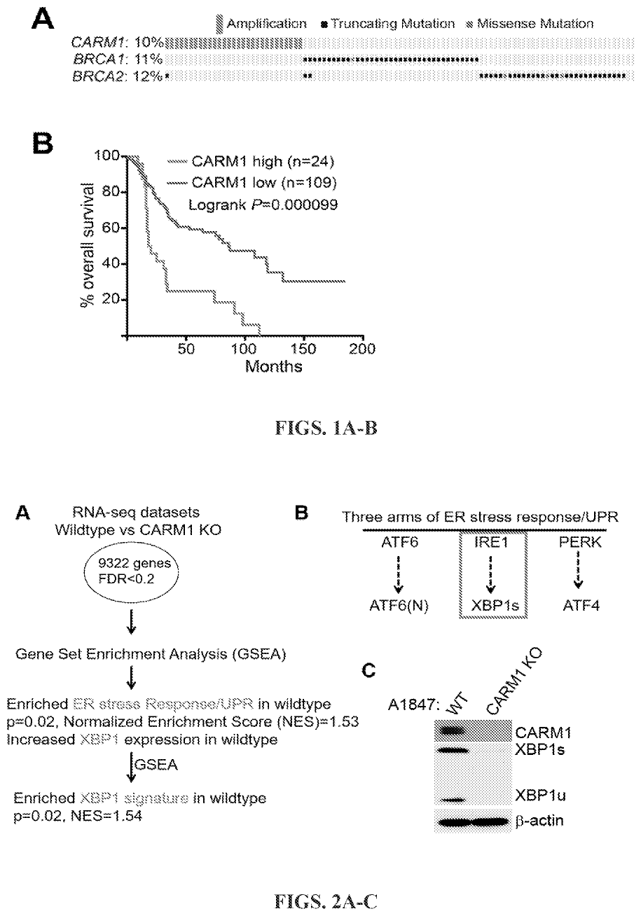

To explore the oncogenic functions of CARM1, the inventors profiled global changes in gene expression by performing RNA-seq analysis in CARM1-high A1847 and the matched isogenic CARM1 knockout A1847 cells. Gene set enrichment analysis (GSEA) (Subramanian et al., 2005) analysis of the differentially expressed genes revealed that ER stress response/Unfolded Protein Response (UPR) signature is significantly enriched in wild-type but not CARM1 knockout cells (FIG. 2A). UPR is cellular defensive ER stress response pathways that are activated under ER stress to promote the survival of cancer cells (Walter& Ron, 2011). Therefore, the upregulated UPR signature indicates that CARM1-high cells exhibit higher ER stress and require activation of UPR for ER homeostasis to survive.

There are three pathways in UPR: ATF6, IRE-1-XBP-1, and PERK pathways (FIG. 2B). IRE-1 is the only ER stress sensor conserved from yeast to mammals that has cytosolic kinase and endoribonuclease (RNase) domains. Under conditions of ER stress, IRE-1 is activated through dimerization and autophosphorylation. Activated IRE-1 removes 26 nucleotides from unspliced XBP-1 (XBP-1u) mRNA to generate spliced XBP-1 (XBP-1s), producing a functional XBP-1s transcription factor (Yoshida et al., 2001). Among these three pathways, the inventors found that the expression of XBP-1 and its target genes, but not the other two pathways, is significantly higher in CARM1-high comparing to CARM1 knockout cells, indicating the activation of the IRE-1-XBP-1 pathway in CARM1-high parental cells. Further GSEA analysis using the XBP-1 signature gene set confirmed that CARM1 upregulates the XBP-1 signature (FIG. 2A). To validate the bioinformatic results, the inventors examined the expression of XBP-1 by western blotting (FIG. 2C). Indeed, both active (XBP-1s) and inactive XBP-1 (XBP-1u) are significantly higher in CARM1-high parental cells compared to CARM1 knockout cells, indicating that ER stress response pathway IRE-1-XBP-1 is activated in CARM1-high cells.

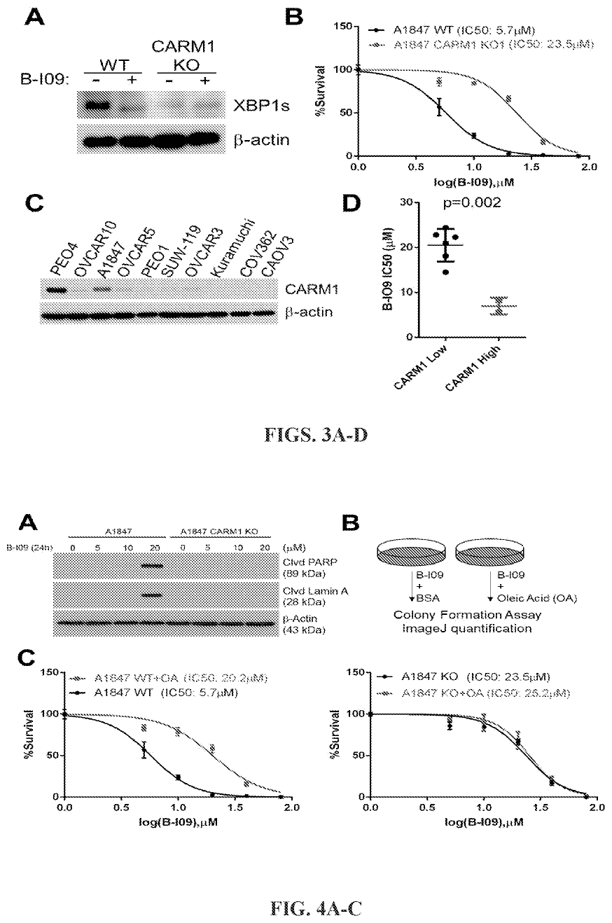

Next, the inventors investigated whether the activated IRE-1-XBP-1 pathway is required for the survival of CARM1-high parental cells. To test this, they used the small molecule inhibitor B-I09 (Tang et al., 2014), which specifically inhibits the IRE-1-XBP-1 pathway by decreasing the expression of active XBP-1s (FIG. 3A). Interestingly, wild-type cells are significantly more sensitive to B-I09 compared with knockout cells (FIG. 3B). This phenotype was further observed in a panel of HGSOC cell lines, where CARM1-high cells have significantly lower IC₅₀ compared to CARM1-low cells (FIGS. 3C-D). To investigate whether the IRE-1-XBP-1 inhibition suppresses CARM1-high cells through inducing cell death, western blotting of apoptotic markers was performed. Upon B-I09 treatment, apoptotic markers such as cleaved PARP and lamin A were observed only in CARM1-high parental but not CARM1 knockout cells, indicating that the inhibition of IRE-1-XBP-1 by B-I09 specifically induces apoptosis in CARM1-high cells (FIG. 4A). Emerging evidence shows that the XBP-1 pathway regulates lipid biosynthesis for cell survival (Sriburi et al., 2004). Therefore, to investigate whether lipid biosynthesis affects the selectivity of CARM1-high cells to inhibit the XBP-1 pathway, cells are treated with B-I09 to inhibit the XBP-1 pathway and rescued with control BSA or oleic acid (OA) (FIG. 4B), which is the upstream product for lipid biosynthesis (Yao et al., 2017). Interestingly, preliminary results showed that oleic acid can rescue A1847 wild-type cells from the inhibition of XBP-1 but has no effect on CARM1 knockout cells (FIG. 4C). This result indicates that the suppression of CARM1-high cells by XBP-1 inhibition depends on lipid biosynthesis. Taken together, these data suggest that the sensitivity to ER stress inhibition is a unique and exploitable therapeutic vulnerability in CARM1-high EOCs.

### REFERENCES FOR EXAMPLE 1

Siegel, R. L., Miller, K. D. & Jemal, A. Cancer statistics, 2019. CA. Cancer J. Clin. 69, 7-34 (2019). PMID: 30620402
Pujade-Lauraine, E., Hilpert, F., Weber, B., Reuss, A., Poveda, A., Kristensen, G., Sorio, R., Vergote, I., Witteveen, P., Bamias, A., Pereira, D., Wimberger, P., Oaknin, A., Mirza, M. R., Follana, P., Bollag, D. & Ray-Coquard, I. Bevacizumab combined with chemotherapy for platinum-resistant recurrent ovarian cancer: The AURELIA open-label randomized phase III trial. J. Clin. Oncol. 32, 1302-1308 (2014). PMID: 24637997
Karakashev, S., Zhu, H., Wu, S., Yokoyama, Y., Bitler, B. G., Park, P.-H., Lee, J.-H., Kossenkov, A. V., Gaonkar, K. S., Yan, H., Drapkin, R., Conejo-Garcia, J. R., Speicher, D. W., Ordog, T. & Zhang, R. CARM1-expressing ovarian cancer depends on the histone methyltransferase EZH2 activity. Nat. Commun. 9, 631 (2018). PMID: 29434212
Cancer Genome Atlas Research Network. Integrated genomic analyses of ovarian carcinoma. Nature 474, 609-615 (2011). PMID: 21720365
Yadav, N., Lee, J., Kim, J., Shen, J., Hu, M. C.-T., Aldaz, C. M. & Bedford, M. T. Specific protein methylation defects and gene expression perturbations in coactivator-associated arginine methyltransferase 1-deficient mice. Proc. Natl. Acad Sci. 100, 6464-6468 (2003). PMID: 12756295
Subramanian, A., Tamayo, P., Mootha, V. K., Mukherjee, S., Ebert, B. L., Gillette, M. A., Paulovich, A., Pomeroy, S. L., Golub, T. R., Lander, E. S. & Mesirov, J. P. Gene set enrichment analysis: a knowledge-based approach for interpreting genome-wide expression profiles. Proc. Natl. Acad. Sci. U.S.A 102, 15545-50 (2005). PMID: 16199517
Walter, P. & Ron, D. The Unfolded Protein Response: From Stress Pathway to Homeostatic Regulation. Science (80-.). 334, 1081-1086 (2011). PMID: 22116877
Yoshida, H., Matsui, T., Yamamoto, A., Okada, T. & Mori, K. XBP-1 mRNA is induced by ATF6 and spliced by IRE-1 in response to ER stress to produce a highly active transcription factor. Cell 107, 881-91 (2001). PMID: 11779464
Tang, C.-H. A., Ranatunga, S., Kriss, C. L., Cubitt, C. L., Tao, J., Pinilla-Ibarz, J. A., Del Valle, J. R. & Hu, C.-C. A. Inhibition of ER stress-associated IRE-1/XBP-1 pathway reduces leukemic cell survival. J. Clin. Invest. 124, 2585-2598 (2014). PMID: 24812669
Sriburi, R., Jackowski, S., Mori, K. & Brewer, J. W. XBP-1: A link between the unfolded protein response, lipid biosynthesis, and biogenesis of the endoplasmic reticulum. J. Cell Biol. 167, 35-41 (2004). PMID: 15466483
Yao, D., Luo, J., He, Q., Shi, H., Li, J., Wang, H., Xu, H., Chen, Z., Yi, Y. & Loor, J. J. SCD1 Alters Long-Chain Fatty Acid (LCFA) Composition and Its Expression Is Directly Regulated by SREBP-1 and PPARY 1 in Dairy Goat Mammary Cells. J. Cell. Physiol. 232, 635-649 (2017). PMID: 27341271
Drew, A. E. Moradei. O, Jacques. SL, Rioux. N, Sjodin. A, Allain. C, Scott. M, Jin. L, Raimondi. A, Handler. J, Ott. H, Kruger. R, McCabe. M, Sneeringer. C, Riera. T, Shapiro. G, Waters. N, Mitchell. L, Duncan. K, Moyer. M, Copeland. R, Smith. J, Chesworth. R, Ribich. S. Identification of a CARM1 Inhibitor with Potent In Vitro and In Vivo Activity in Preclinical Models of Multiple Myeloma. Sci. Rep. 7, 17993 (2017). PMID: 29269946
Liu, J., Cinar, R., Xiong, K., Godlewski, G., Jourdan, T., Lin, Y., Ntambi, J. M. & Kunos, G. Monounsaturated fatty acids generated via stearoyl CoA desaturase-1 are endogenous inhibitors of fatty acid amide hydrolase. Proc. Natl. Acad. Sci. 110, 18832-18837 (2013). PMID: 24191036

### Example 2-ARID1A

ARID1A is a DNA-interacting protein subunit of the SWI/SNF chromatin remodeling complex and functions as a tumor suppressor. ARID1A is mutated in more than 50% of ovarian clear cell carcinoma (OCCC) cases resulting in its loss of expression in ˜90% mutated cases. Loss of ARID1A is frequently associated with chromatin remodeling dysfunction, tumor growth, and poor patient prognosis. There is an unmet clinical need in treating ARID/A-mutant ovarian cancers because standard of care therapies, such as platinum-based chemotherapies, have failed. Thus, understanding the mechanisms that govern ARID/A-mutant tumor growth may provide new therapeutic avenues for OCCC treatment. This is especially important because OCCC has the worst prognosis of all ovarian cancer subtypes in its advanced stages. The overall goal of this proposal is to explore a new targeted approach for the treatment of ARID/A-mutant ovarian cancers.

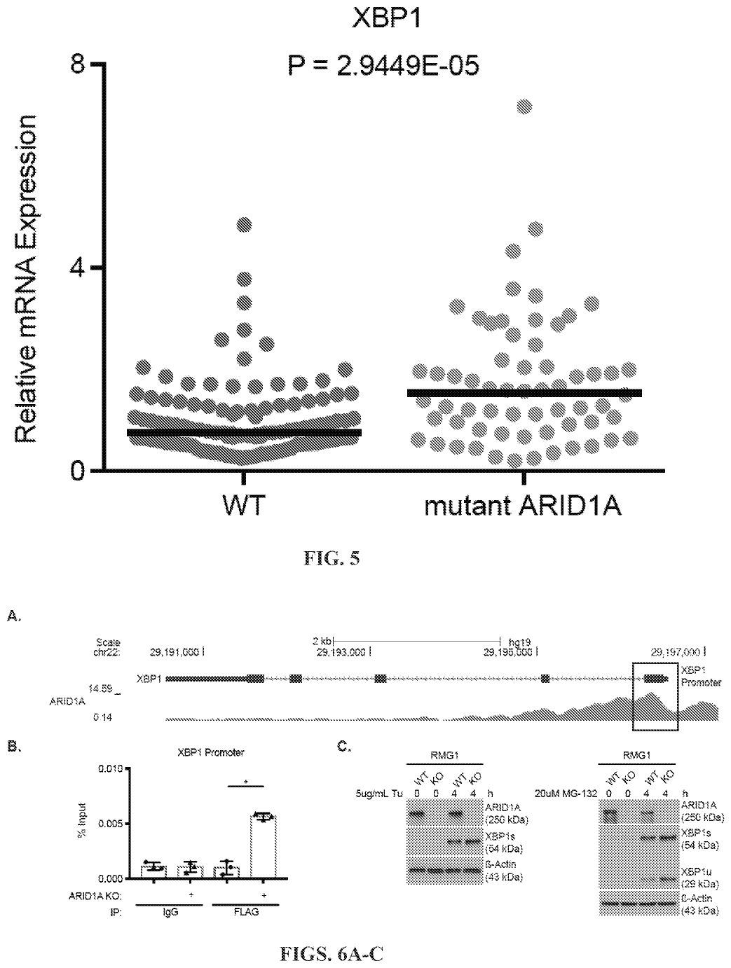

To determine how ARID1A is implicated in ER stress, the inventors analyzed the TCGA database to determine a link between ARID1A mutation and the ER stress response. OCCCs are not included in the TCGA database. The only available datasets exhibiting high percentages of ARID1A-mutation, relatable to ARID1A-mutated OCCCs, were in endometrial uterine carcinomas. This is because there is evidence to suggest that endometriosis is associated with the development of both OCCC and endometrial uterine carcinomas (Fukumoto el al., 2018; Ayhan et al., 2012). The inventors found that XBP-1 mRNA expression is significantly enhanced in ARID1A-mutant uterine tumors compared to uterine tumors possessing wild-type ARID1A (FIG. 5). Unbiased ChIP-seq was performed to determine ARID1A interactions with ER stress response genes in the RMG1 ARID1A wild-type OCCC cell line. ARID1A ChIP-seq and ChIP RT-qPCR validation shows ARID1A interaction with the XBP-1 gene promoter (FIGS. 6A-B). To validate if ARID1A loss results in changed XBP-1 expression under ER stress conditions, the inventors performed western blots in ARID1A wild-type parental control and CRISPR/Cas9 ARID1A KO RMG1 cells. An upregulation of unspliced and spliced XBP-1 can be seen via western blot under ER stress induction through tunicamycin (Tu) and MG132 treatments (FIG. 6C). Spliced XBP-1 represents the transcription factor that regulates ER stress response target genes. Unspliced XBP-1 is representative of XBP-1 gene transcription. These experiments suggest that ARID1A interacts with and regulates XBP-1 gene expression under ER stress conditions. Increased unspliced XBP-1 protein further supports that ARID1A regulates XBP-1 gene expression transcriptionally and is independent of IRE-1 splicing. More experimentation will be performed to determine the mechanism by which the SWI/SNF complex regulates ER stress in OCCC.

To examine the effects of ARID1A status on the ER stress response, the inventors plan to treat RMG1 wild-type and CRISPR/Cas9 knock-out ARID1A cells with the ER stress inducing agents such as dithiothreitol (DTT), MG132, tunicamycin (Tu), and thapsigargin (Tg) (Oslowski and Urano, 2011). The inventors will expand this analysis into a panel of ARID1A wild-type (such as RMG1, OVCA429 and KK) and mutant (such as TOV21G, OVTOKO and OVISE) OCCC cell lines to further establish the correlation between ER stress response and ARID1A mutational status. To compliment the results using these inhibitors, the inventors will use an sh-XBP-1 inducible knock-down system to genetically establish the importance of the XBP-1 ER stress pathway in promoting survival in ARID1A mutant/CRISPR-Cas9 knock-out ARID1A cells. They expect that genetic suppression of XBP-1 expression will promote apoptosis in ARID1A mutant/CRISPR-Cas9 knock-out, but not wild-type OCCC cell lines. Apoptosis will be analyzed by western blot of cleaved PARP and cleaved caspase 3. They saw an increase of XBP-1 expression when ARID1A was lost but other ER stress responses may contribute to cell survival.

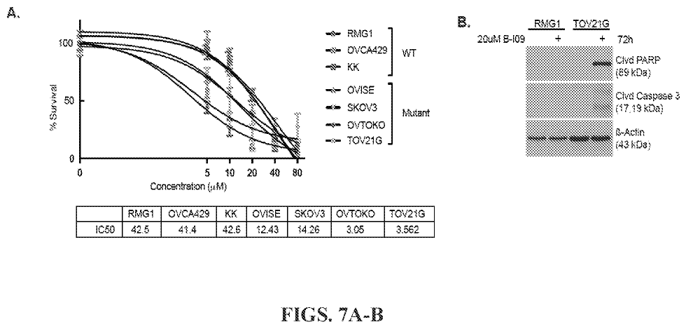

To test this, ER stress response proteins, such as PERK, ATF4, ATF6 and BiP will be assessed under these treatments via western blot and RT-qPCR to determine their contributions to ARID1A loss and OCCC. All experimentation will be performed in triplicate to ensure the data is reproducible and that changes are statistically significant. Based upon preliminary data from FIGS. 5 and 6A-C, the inventors have performed colony formation assays to show that, compared with ARID1A wild-type OCCC cells, ARID1A-mutant OCCC cells are more sensitive to ER stress response inhibition via B-I09 treatment (FIGS. 7A-B). This shows that XBP-1 is important for maintaining the survival of ARID1A mutant cells. Together, these experiments will establish a link between ARID1A and ER stress in OCCC.

The inventors have shown that ARID1A interacts with promoter regions of the XBP-1 ER stress response gene. It is not known if ARID1A's loss of expression alters the occupancy of the SWI/SNF complex to ER stress response genes. It is also not known what mechanisms cause increased XBP-1 expression when ARID1A is lost from its gene promoter. However, the inventors hypothesized that the ARID1A-containing SWI/SNF complex represses XBP-1 gene transcription by preventing the association of the transcription activator ATF6 with the XBP-1 promoter during ER stress.

Preliminary data supports the hypothesis that the SWI/SNF complex regulates ER stress in RMG1 cells through XBP-1 gene interactions (FIG. 6A). To confirm ARID1A involvement in ER stress regulation, the inventors will expand both ChIP and expression analysis in RMG1 CRISPR/Cas9 ARID1A knock-out cells and OCCC cells possessing mutant ARID1A, such as OVISE, SKOV3, OVTOKO, and TOV21G with or without wild-type ARID1A restoration. In addition, I will perform ChIP analysis of active epigenetic markers such as lysine 27 acetylated histone H3 (H3K27ac) and lysine 4 trimethylated histone H3 (H3K4me3) and repressive epigenetic markers such as lysine 9 di- or trimethylated histone H3 (H3K9me2/3) on the XBP-1 promoter to determine how epigenetic status of the XBP-1 gene promoter is affected under ER stress with or without ARID1A expression.

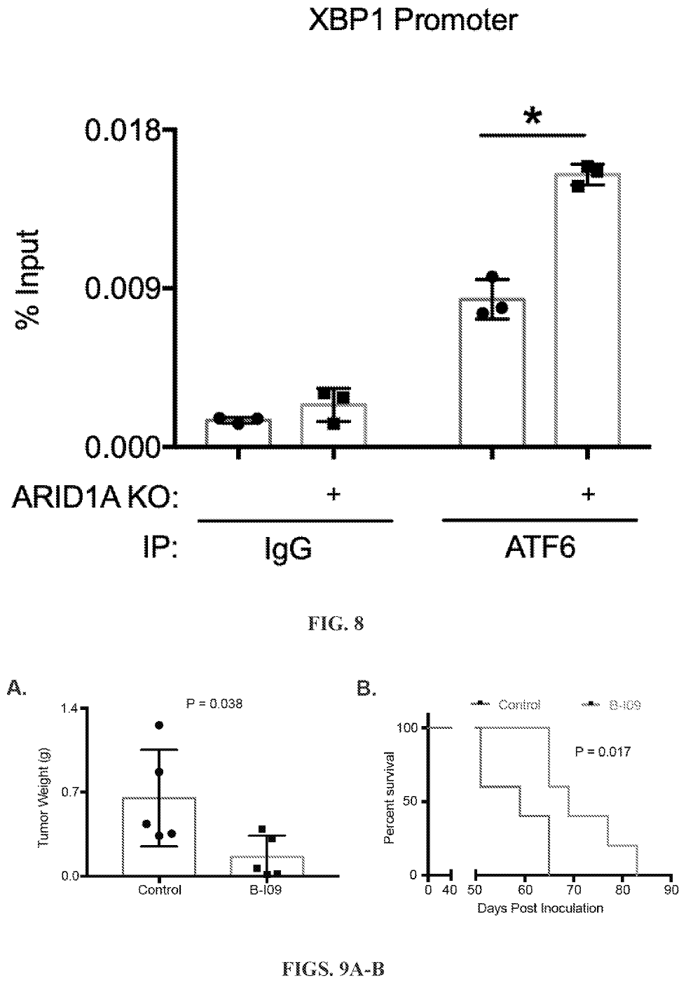

Based upon western blot analyses, the time points in which XBP-1 protein levels increase under differing ER stressors, such as with tunicamycin, will be used to perform ChIP analyses of these epigenetic marks. To determine if ARID1A is guiding the SWI/SNF complex to regulate XBP-1 gene expression, SNF5 (a core SWI/SNF protein subunit) ChIP will be performed. ARID1A loss may promote other transcription factors to enhance XBP-1 expression. Preliminary data shows that TF6, known to interact with and regulate XBP-1 expression (Yoshida el al., 2001), interacts with the ABP-1 promoter more in RMG1 cells lacking ARID1A expression (FIG. 8). To further elucidate the roles by which ATF6 is regulating XBP-1 expression, the inventors will confirm ATF6 localization to the nucleus to regulate XBP-1. They will perform cellular fractionation, separating the nucleus from the cytosol, to show ATF6 translocation to the nucleus under previously mentioned ER stress conditions. They will also knockdown ATF6 expression, through shRNA production, to determine its role within XBP-1 gene regulation under ER stress conditions and expect that ATF6 suppression will reduce XBP-1 expression in ARID1A knock-out cells compared to cells with wild-type ARID1A under tunicamycin and thapsigargin induced ER stress. These experiments will be repeated three times to ensure reproducibility and statistical significance. Together, these experiments will determine if ARID1A mediates regulation of XBP-1 gene expression by the SWI/SNF complex, a link that has never been reported.

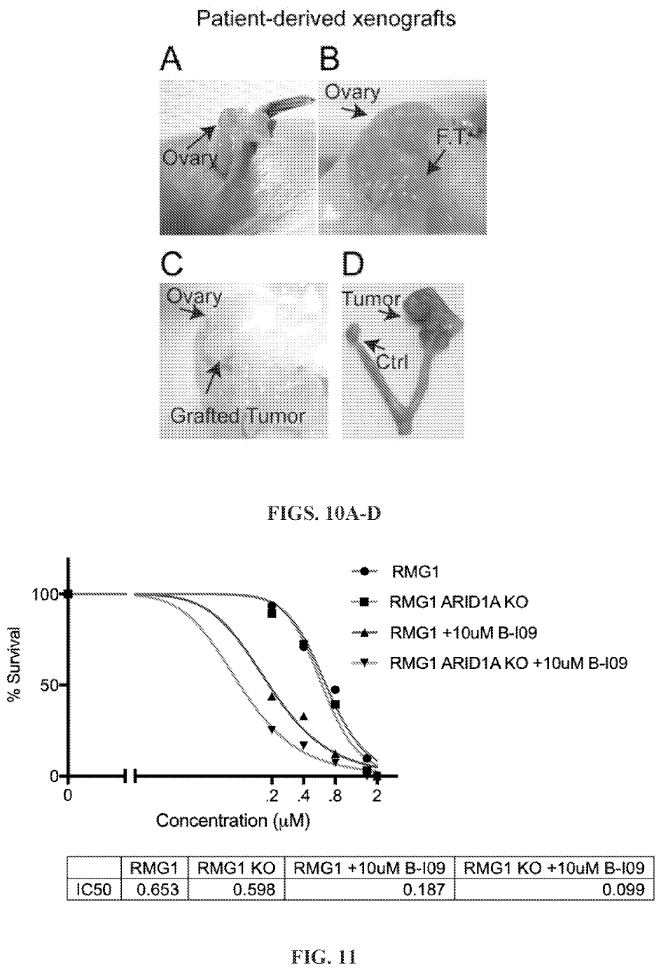

The inventors hypothesize that OCCC tumors possessing mutant ARID1A will be more sensitive to B-I09 treatment. They will exhibit reduced tumor growth. Preliminary data show that treatment of B-I09 reduces OCCC tumor growth in vivo in an established OCCC genetic mouse model driven by conditional inactivation of Arid1a and activation of Pik3ca (Chandler el al., 2015) (FIGS. 9A-B). To examine in vivo B-I09 treatment selectivity in conjunction with ARID1A status, 1×10⁶ cells with wild-type (e.g., RMG1 and OVCA429) and mutant ARID1A (TOV21G and OVISE) OCCC cells will be injected intrabursally into NSG mice (Nacarelli el al., 2019; Fukumoto et al., 2018; Wu et al., 2018; Bitler et al., 2017). Upon surgery recovery, tumor formation will be monitored via palpitations and caliper measurements. The inventors will randomize and treat with vehicle control or 50 mg/Kg of B-I09 intraperitoneally (i.p.) daily for 3 weeks (5 days on/2 off per week) once the tumors are approximate volumes of 100 mm³.

Upon completion of this experiment, immunohistochemistry will be performed on harvested tumors. Staining for Ki67 (proliferation), cleaved caspase 3 (apoptosis), and XBP-1s (suppressed upon B-I09 treatment) will be analyzed. To further validate the efficacy of B-I09 treatment in treating OCCC, the inventors will xenograft de-identified ARID1A-mutant or wild-type patient-derived xenograft (PDX) tumors into the bursa of NSG mice (FIGS. 10A-D). The PDX tumors have been validated for ARID1A mutation and lack ARID1A expression (data not shown). Furthermore, PDX tumors have been successfully type and two ARID1A mutant OCCC PDXs that are readily available for my experiments. The inventors will treat PDX mice with 50 mg/Kg B-I09 i.p. Together, these experiments will determine if ARID1A mutation in OCCC promotes sensitivity to IRE-1-XBP-1 ER stress response inhibition in vivo. These studies will also establish the ER stress response as a therapeutic target in OCCC in which ARID1A expression is lost.

Resistance to cisplatin has been associated with an increase in ER stress-mediated cellular survival mechanisms in ovarian cancer (Avril et al., 2017; Lei et al., 2015; Xu et al., 2015). These findings suggest that combination with ER stress response inhibitors may sensitize ARID1A-mutant OCCCs that are typically refractory to platinum-based chemotherapy. The inventors hypothesize that ER stress inhibition is synergistic with platinum-based chemotherapy in ARID1A-mutated OCCCs. To test this hypothesis, the inventors will treat ARID1A wild-type, ARID1A CRISPR-Cas9 knock-out, and ARID/A-mutant or wild-type OCCC cells with increasing concentrations of cisplatin or carboplatin. ER stress response induction will be analyzed by western blot and RT-qPCR analyzing the expression of ER stress response proteins such as IRE-1, XBP-1, PERK, ATF6 and BiP. They expect that platinum-based therapy-induces enhanced ER stress responses in ARID1A mutant/knock-out cells. ER stress response induction may sensitize them to treatment combination of platinum-based therapies and B-I09. Preliminary data shows that cisplatin and B-I09 combination treatment reduces the IC₅₀ of cisplatin more effectively in RMG1 CRISPR/Cas9 KO cells compared to their wild-type parental control (FIG. 11). The inventors will expand this analysis into a panel of ARID1A wild-type (such as RMG1, OVCA429 and KK) and mutant (such as TOV21G, OVTOKO, and OVISE) OCCC cell lines to further establish the efficacy of combination treatment of platinum-based therapy and B-I09. The coefficient of interaction (CI), as established by the Chou and Talalay method, will be applied for the analysis of synergism using CompuSyn software. To determine if B-I09 and platinum-based therapy combination induces apoptosis, the inventors will perform western blots to analyze cleaved caspase 3 and cleaved PARP. They expect to see a strong synergy between B-I09 and cisplatin treatment in ARID1A-mutant, but not wild-type OCCC, cell lines. To further test the effect of ARID1A-mutation status on the synergy of B-I09 and platinum-based therapy treatments, they will restore ARID1A expression in ARID1A-mutant cell lines. The inventors expect that ARID1A restoration will reduce the synergy of B-I09 and cisplatin combination treatment. Together, these experiments will show that platinum-based chemotherapy treatment induces ER stress-mediated survival in ARID1A-mutated OCCC, and that ARID1A mutation promotes sensitivity to B-I09 and cisplatin combination treatment.

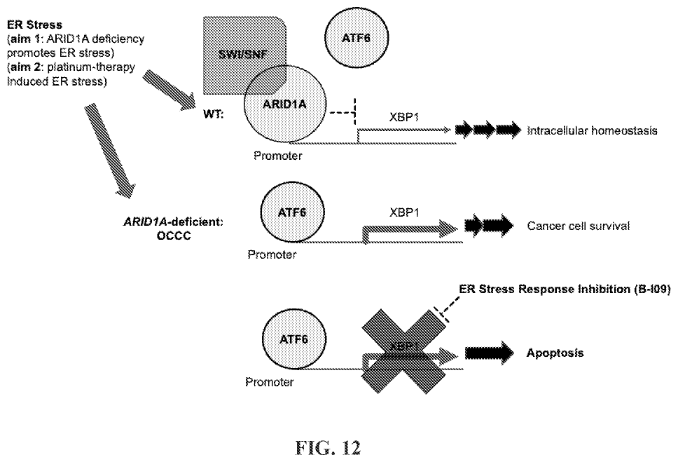

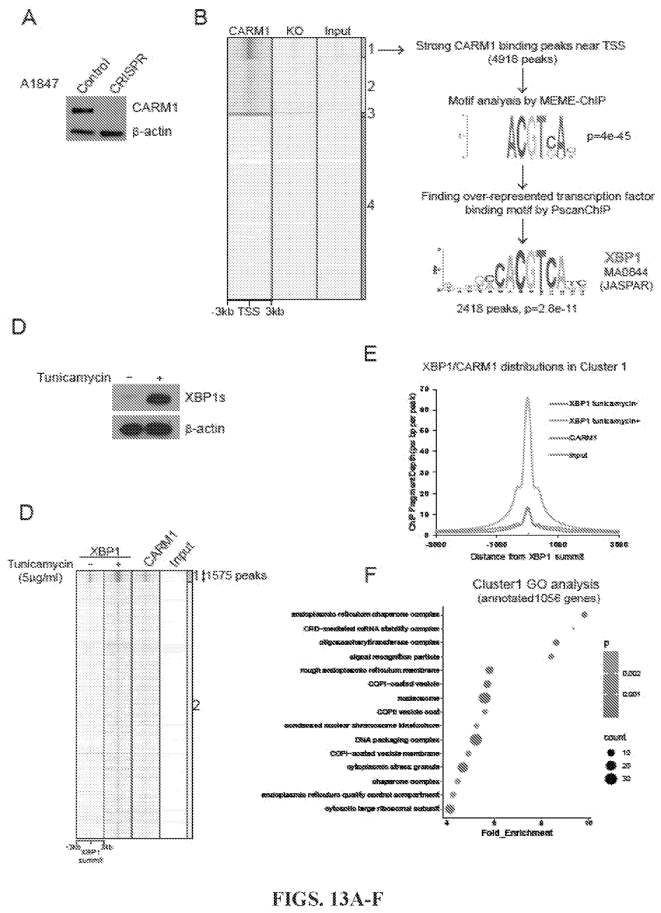

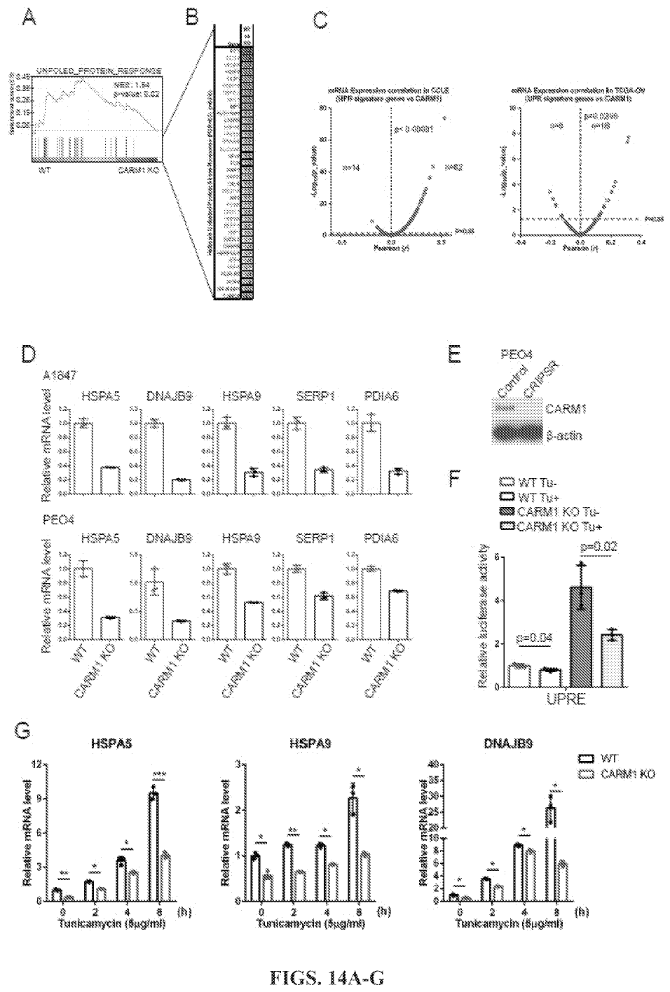

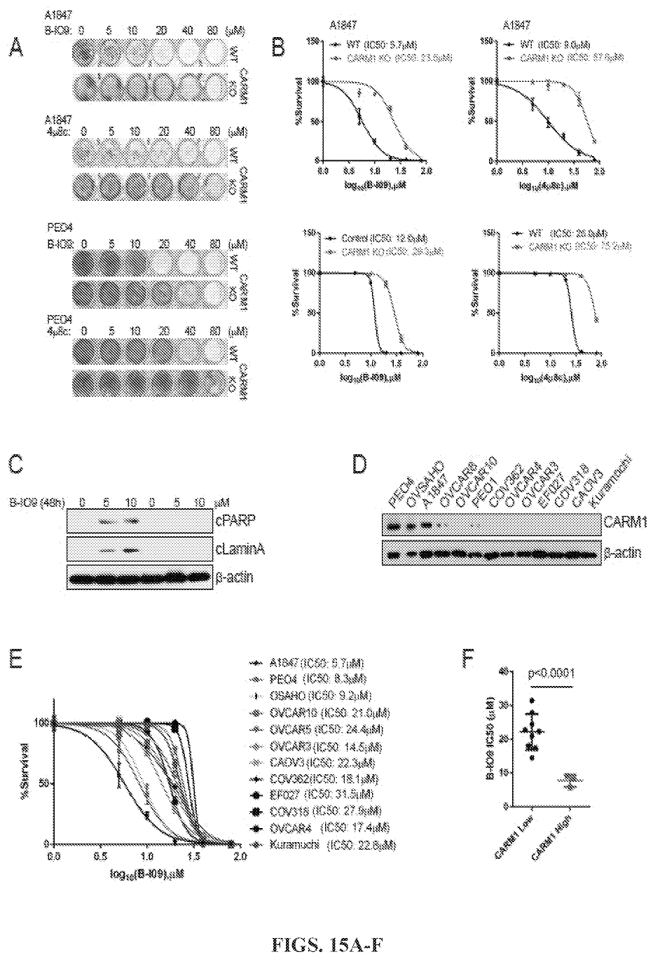

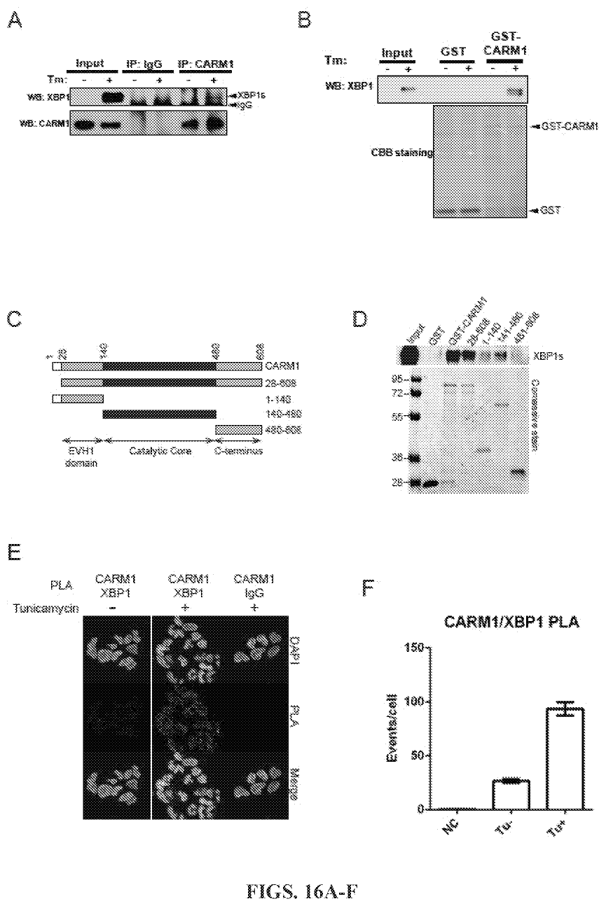

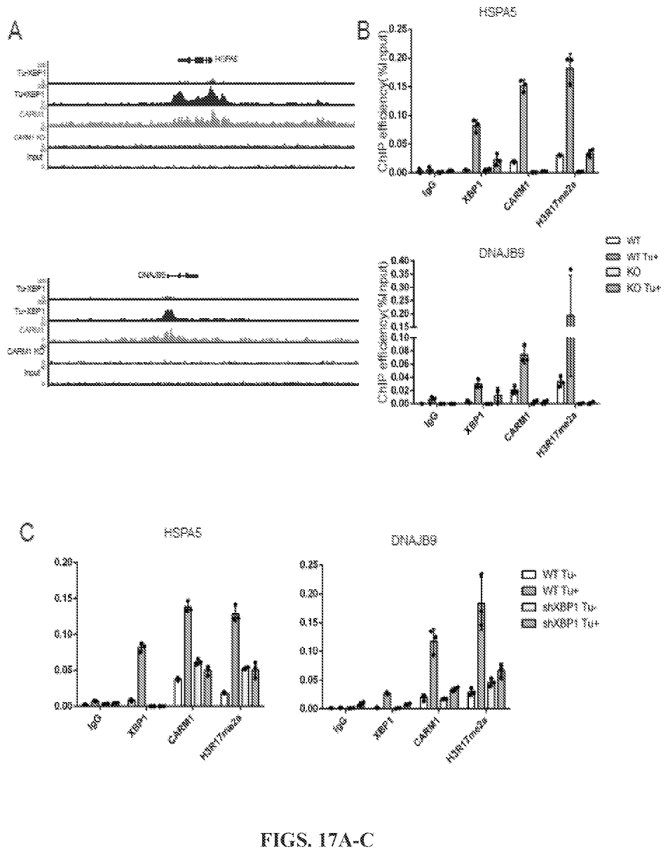

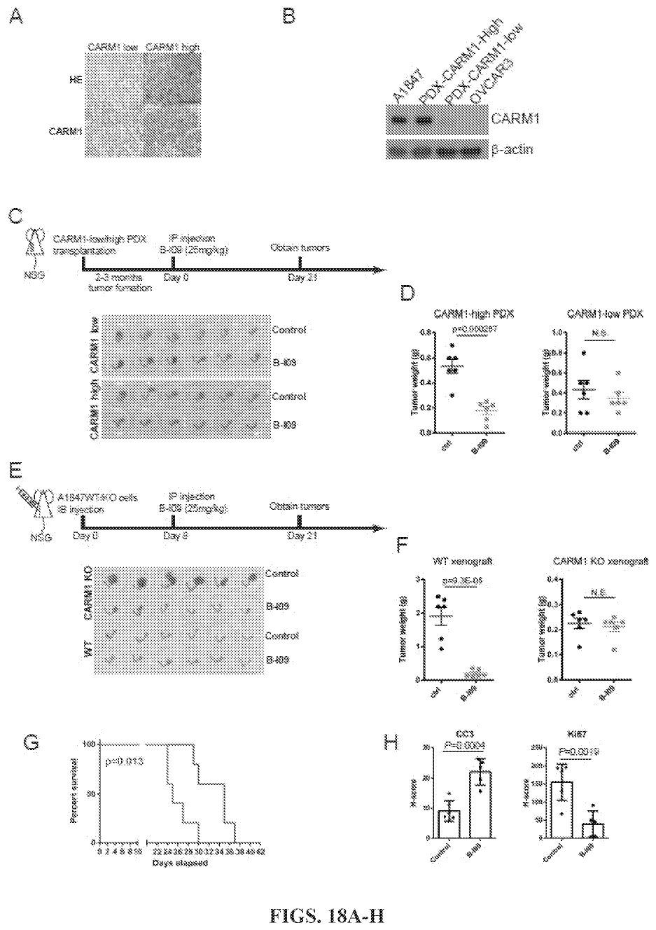

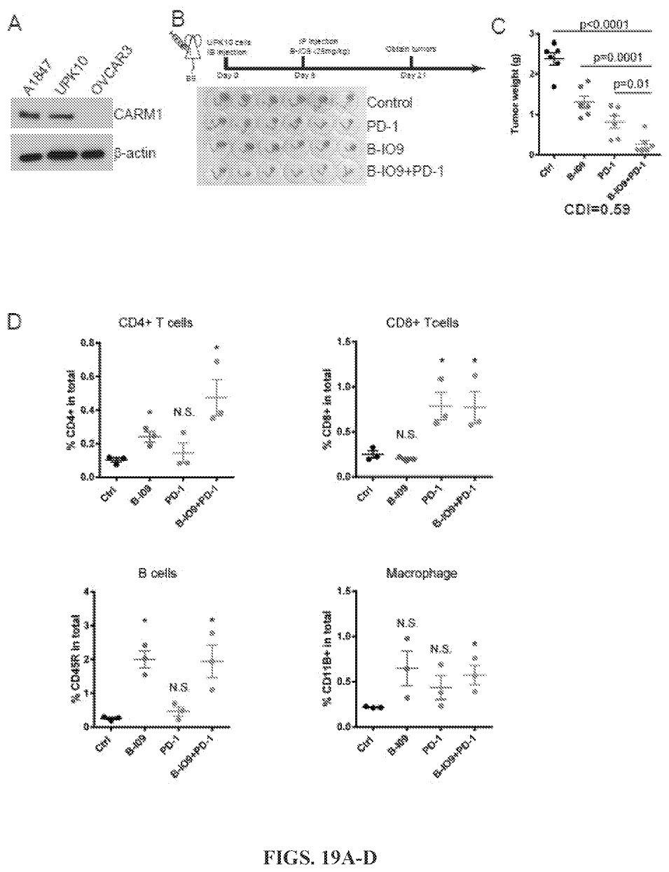

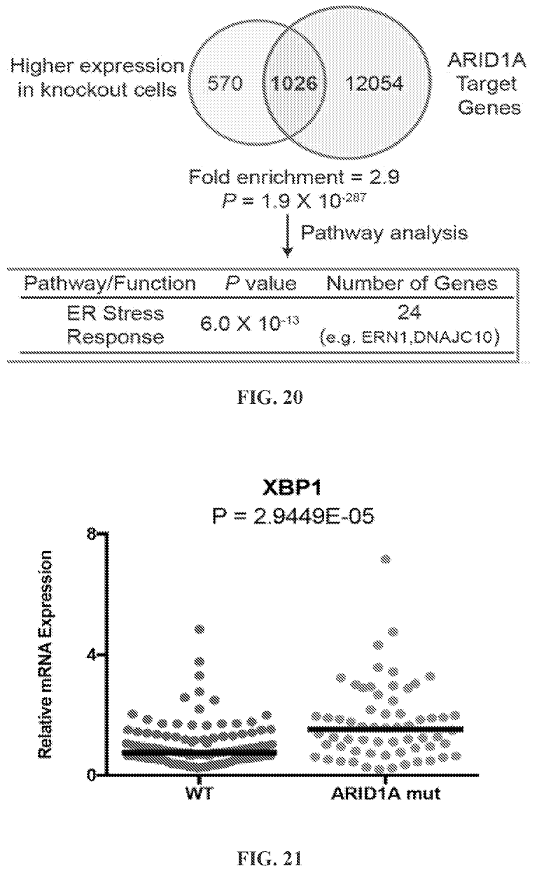

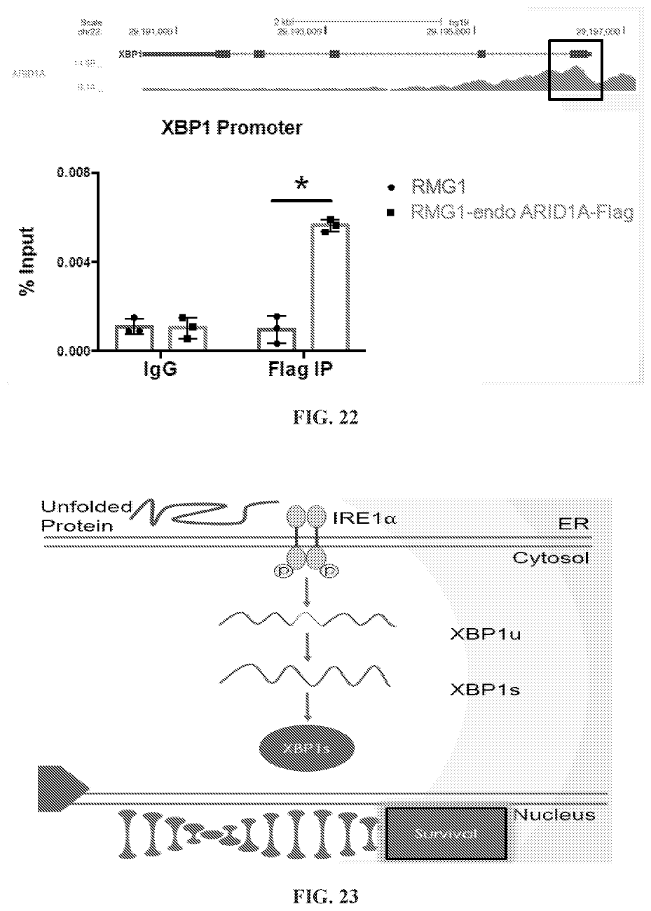

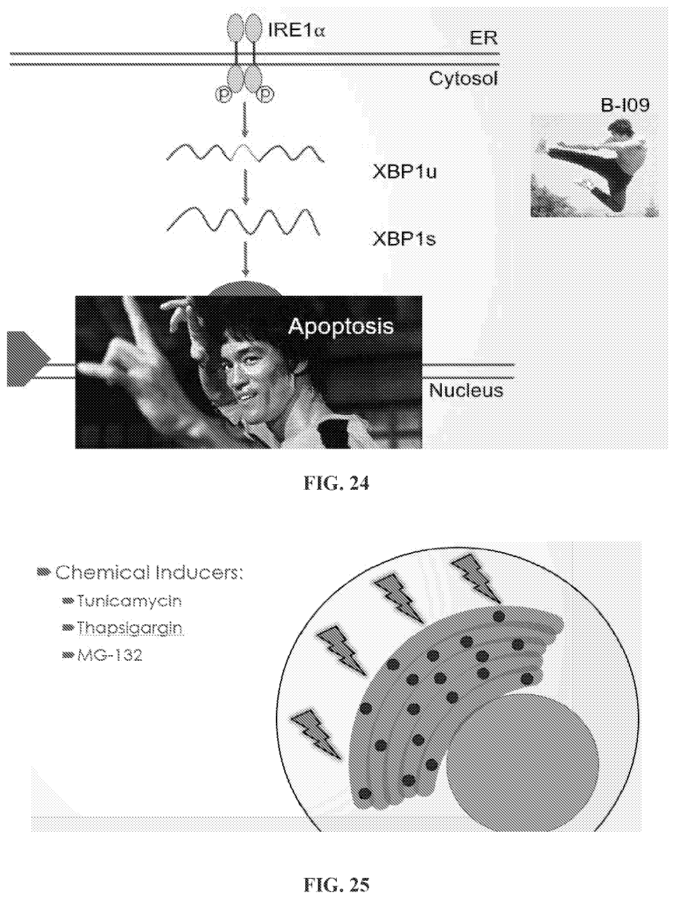

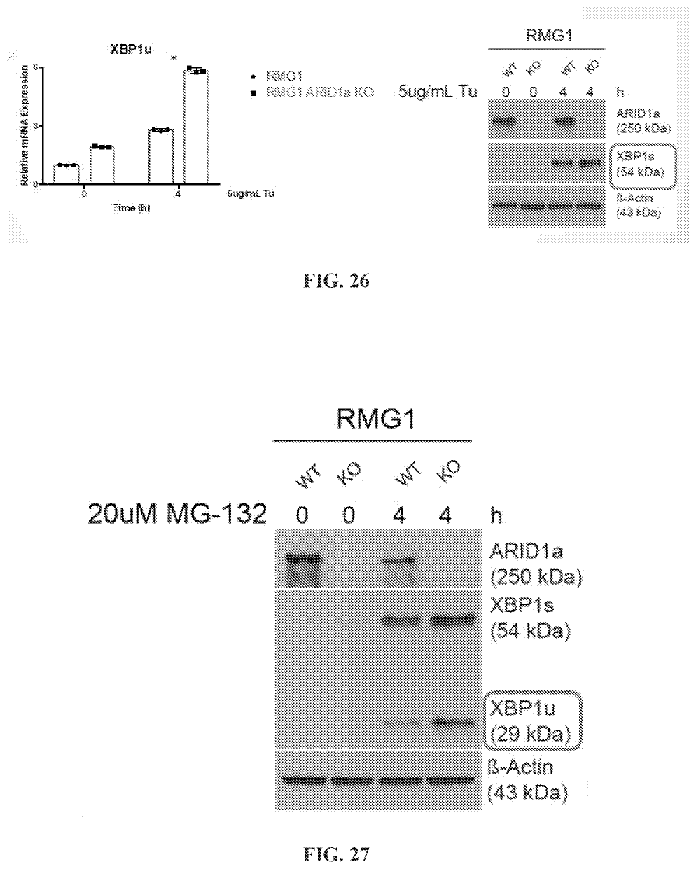

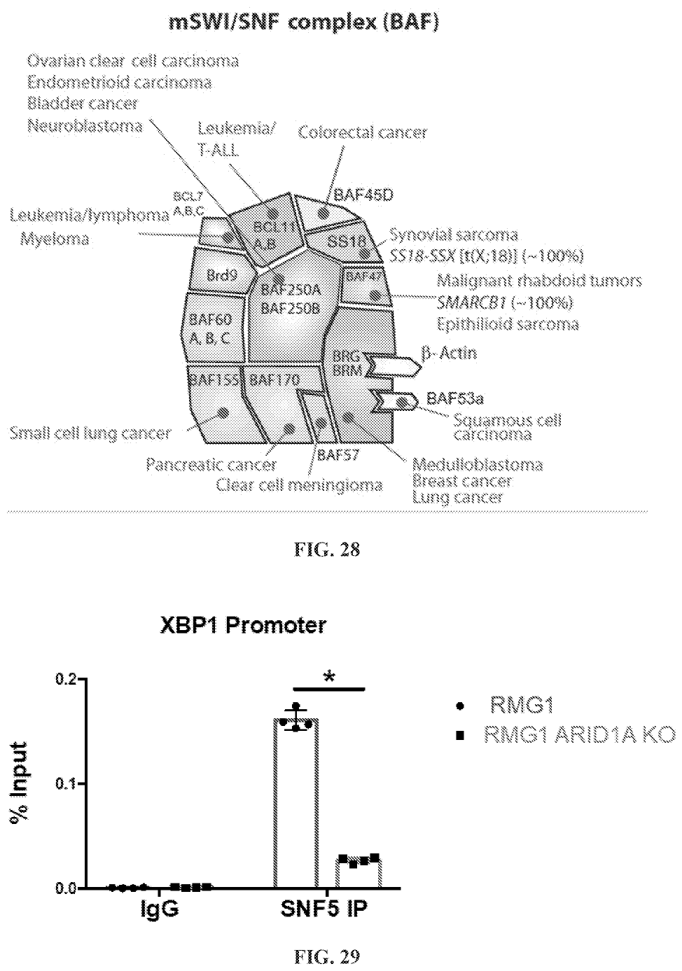

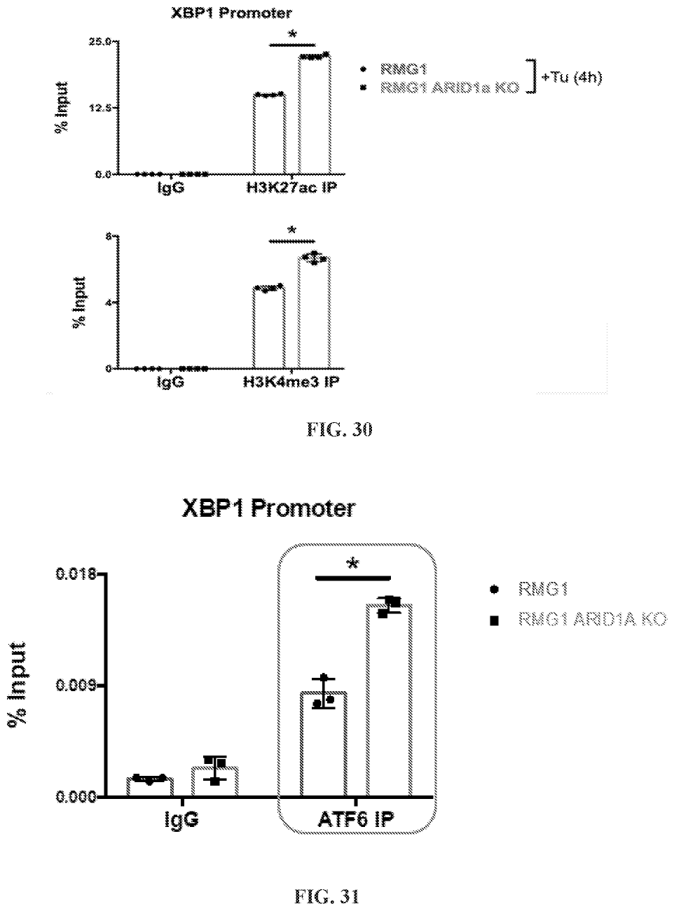

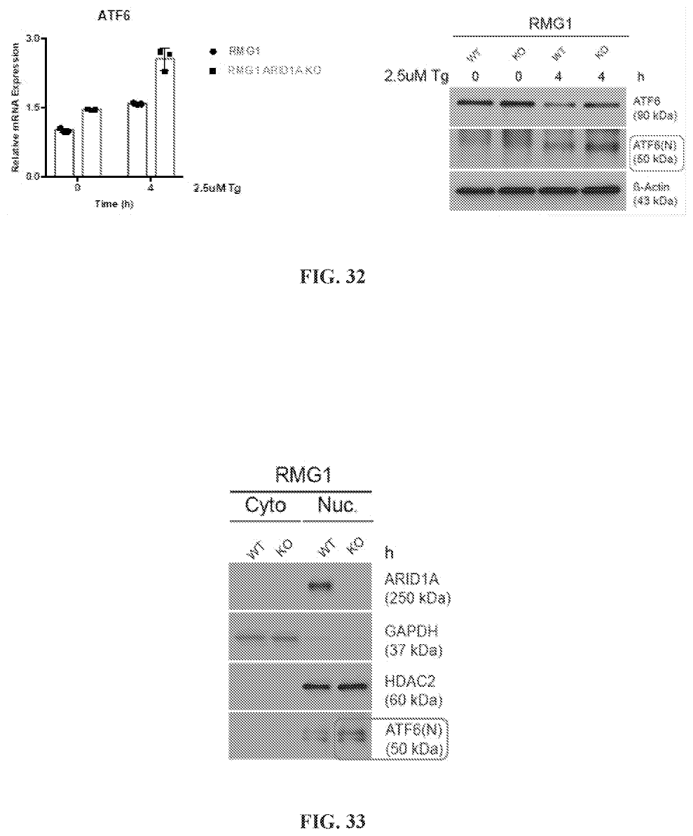

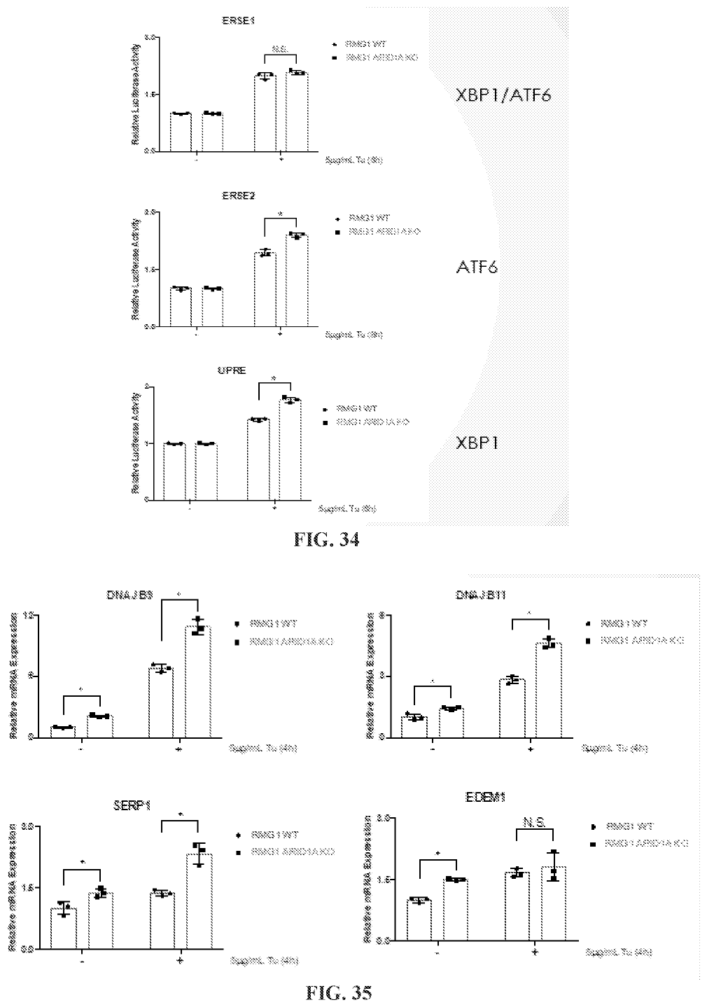

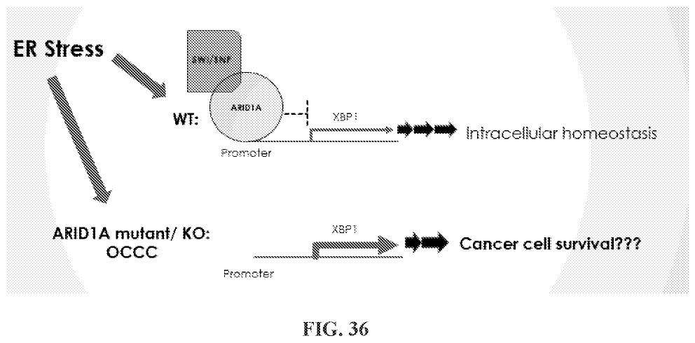

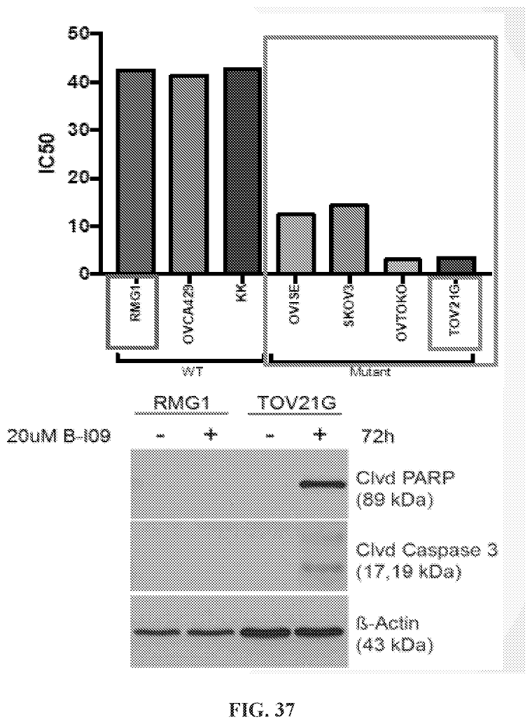

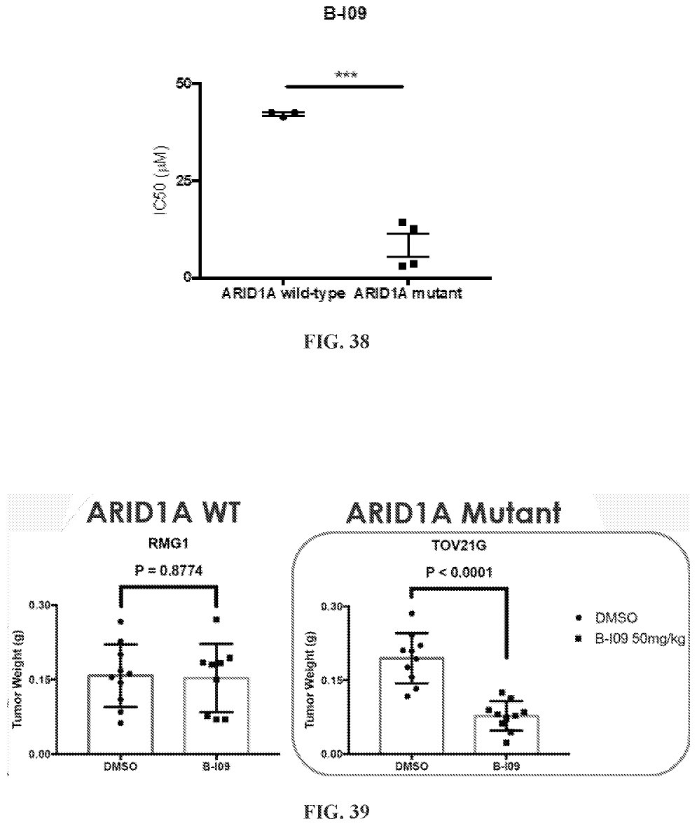

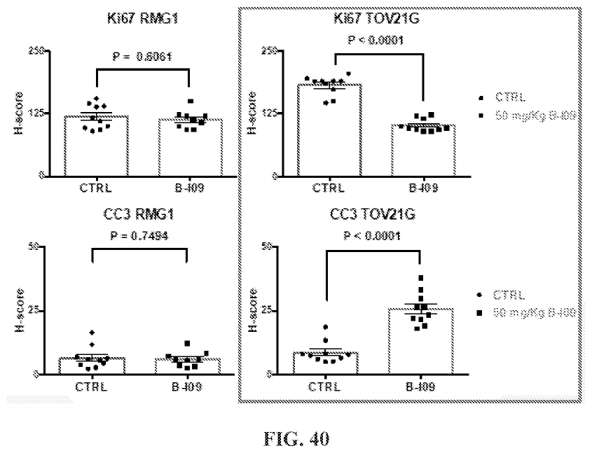

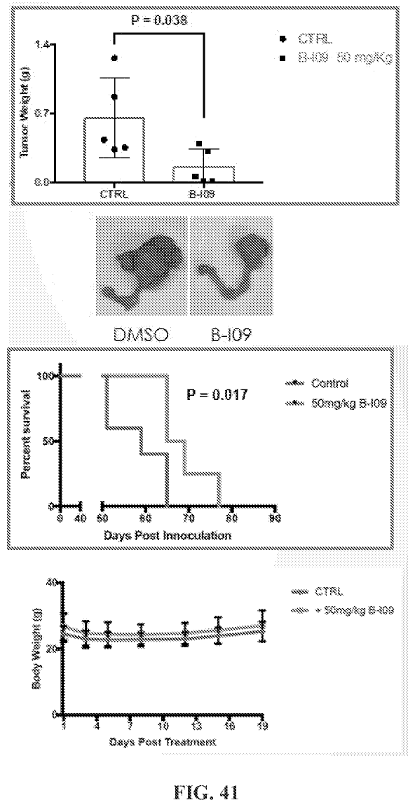

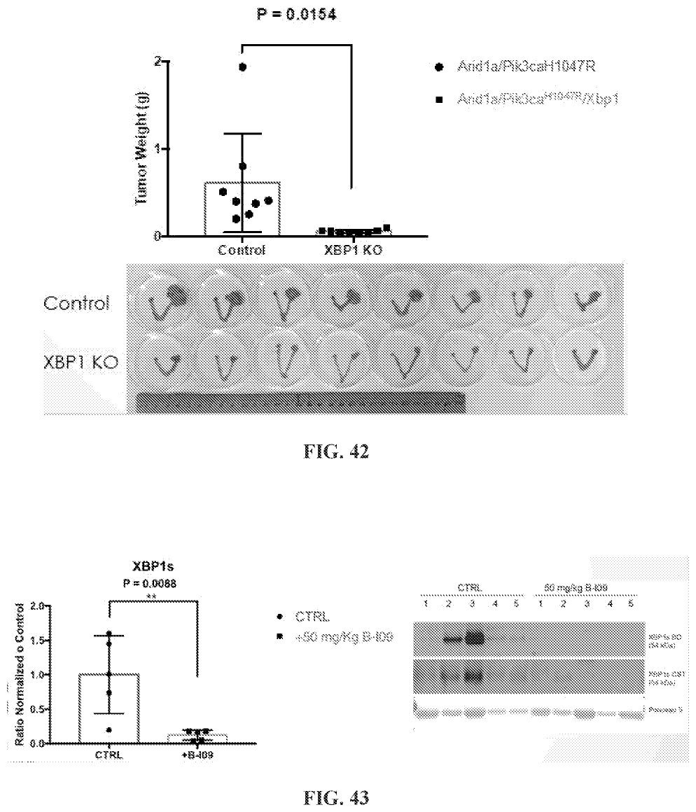

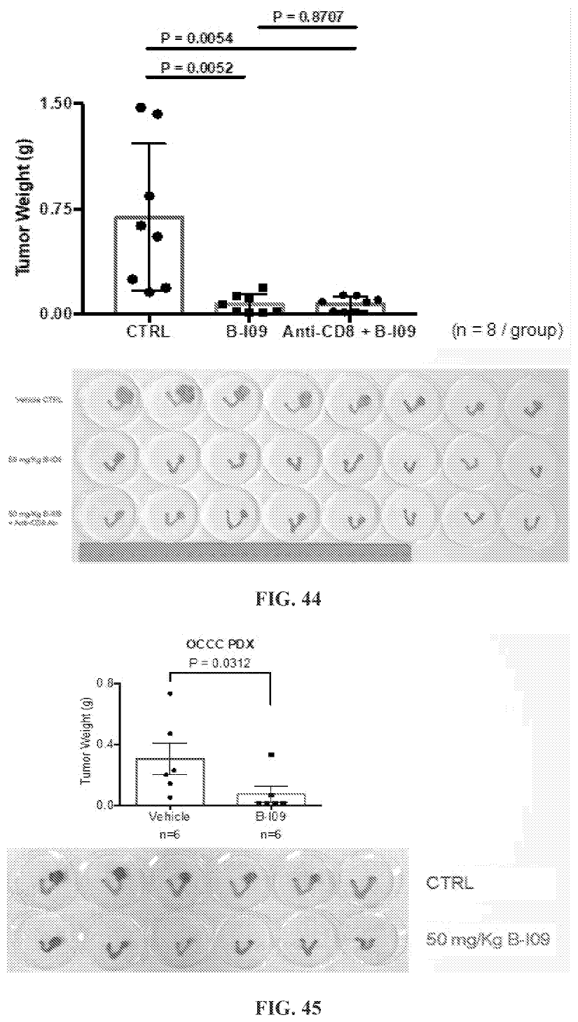

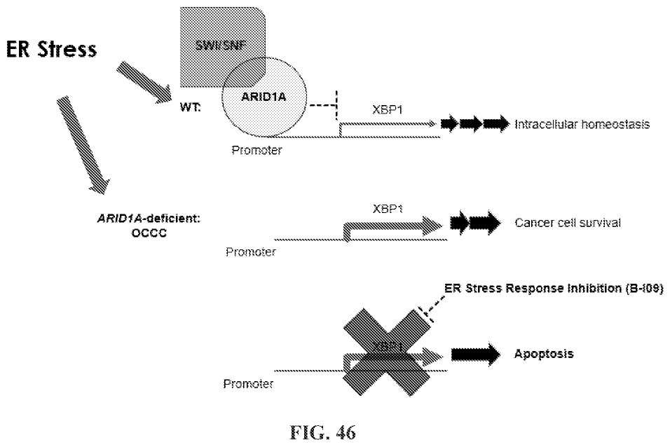

The inventors hypothesize that platinum and B-I09 ER stress response inhibition are synergistic in reducing tumor growth and dissemination into the peritoneal cavity and increasing survival, in an ARID1A status dependent manner, as determined by tumor mass and Kaplan-Meier analysis, respectively. To examine this, the Arid1a^{flox/flox}; (Gt)Rosa26Pik3ca*^{H1047R} OCCC mouse model will be employed. Mice will be randomized prior to drug treatments. 50 mg/Kg B-I09 (5 days on/2 days off) and a 5 mg/Kg cisplatin (once a week) i.p. injection treatment regimen will be administered for three weeks. Single treatment of B-I09, or cisplatin, mice will be compared to a combination group and a vehicle treated group. Upon completion of this experiment, immunohistochemistry will be performed on harvested tumors. Staining for Ki67 (proliferation), and cleaved caspase 3 (apoptosis) will be analyzed. The inventors will also use the orthotopic NSG and PDX models of OCCC with wild-type or mutant ARID1A, to test whether the selectivity of B-I09 and cisplatin combination in vivo is ARID1A status dependent (FIG. 12). Together, these experiments will reveal if ARID1A inactivation enhances ER stress induced by platinum-based chemotherapies in OCCCs. They will also establish if the use of ER stress response inhibition in combination with platinum-based chemotherapies is a potential therapeutic strategy for ARID1A-mutated OCCCs.

### REFERENCES FOR EXAMPLE 2

Fukumoto et al., 2018. SWI/SNF complexes in ovarian cancer: mechanistic insights and therapeutic implications. Molecular Cancer Research, 16(12), pp. 1819-1825. PMID: 30037854; PMCID: PMC6279546 [Available on 2019 Dec. 1]; DOI: 10.1158/1541-7786.MCR-18-0368.
Ayhan et al., 2012. Loss of ARID1A expression is an early molecular event in tumor progression from ovarian endometriotic cyst to clear cell and endometrioid carcinoma. International journal of gynecological cancer: official journal of the International Gynecological Cancer Society, 22(8), p.1310. PMID: 22976498; PMCID: PMC3460070; DOI: 10.1097/IGC.0b013e31826b5dcc.
Chandler el al., 2015. Coexistent ARID1A-PIK3CA mutations promote ovarian clear—cell tumorigenesis through pro-tumorigenic inflammatory cytokine signalling. Nature communications, 6, p.6118. PMID: 25625625; PMCID: PMC4308813; DOI: 10.1038/ncomms7118.
Avril et al., 2017. Endoplasmic reticulum stress signaling and chemotherapy resistance in solid cancers. Oncogenesis, 6(8), p.e373. PMID: 28846078; PMCID: PMC5608920; DOI: 10.1038/oncsis.2017.72.
Lei et al., 2015. Inhibition of ANKRD1 sensitizes human ovarian cancer cells to endoplasmic reticulum stress-induced apoptosis. Oncogene, 34(4), p.485. PMID: 24531715; DOI: 10.1038/onc.2013.566.
Xu et al., 2015. Tolerance to endoplasmic reticulum stress mediates cisplatin resistance in human ovarian cancer cells by maintaining endoplasmic reticulum and mitochondrial homeostasis. Oncology reports, 34(6), pp.3051-3060. PMID: 26398138; DOI: 10.3892/or 2015.4283.
Oslowski, C. M. and Urano, F., 2011. Measuring ER stress and the unfolded protein response using mammalian tissue culture system. In Methods in enzymology (Vol. 490, pp. 71--92). Academic Press. PMID: 21266244; PMCID: PMC3701721; DOI: 10.1016/B978-0-12-385114-7.00004-0.
Yoshida el al., 2001. XBP-1 mRNA is induced by ATF6 and spliced by IRE-1 in response to ER stress to produce a highly active transcription factor. Cell, 107(7), pp. 881-891. PMID: 11779464.
Nacarelli et al., 2019. NAD+ metabolism governs the proinflammatory senescence—associated secretome. Nat Cell Biol. 21(3), pp. 397-407. PMID: 30778219. PMCID: in process.
Fukumoto et al., 2018. Repurposing Pan—HDAC inhibitors for ARID1A—mutated ovarian cancer. Cell reports, 22(13), pp. 3393-3400. PMID: 29590609; DOI: 10.1016/j.celrep.2018.03.019.
Wu et al., 2018. SWI/SNF catalytic subunits' switch drives resistance to EZH2 inhibitors in ARID1A-mutated cells. Nature communications, 9. PMID: 30297712; DOI: 10.1038/s41467-018-06656-6
Bitler et al., 2017. ARID1A—mutated ovarian cancers depend on HDAC6 activity. Nature cell biology, 19(8), p.962. PMID: 28737768; DOI: 10.1038/ncb3582.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 5,538,880
U.S. Pat. No. 5,928,906
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Brown et al., J. Immunol. Meth., 12; 130(1): 111-121, 1990.
De Jager et al., Semin. Nucl. Med. 23(2), 165-179, 1993.
Doolittle and Ben-Zeev, Methods Mol. Biol., 109, 215-237, 1999.
Gulbis and Galand, Hum. Pathol. 24(12), 1271-1285, 1993.
Nakamura et al., In: Enzyme Immunoassays: Heterogeneous and Homogeneous Systems, Chapter 27, 1987.
Sambrook et al., In: Molecular cloning: a laboratory manual, 2^{d} Ed., Cold Spring Harbor Laboratory Press, Cold Spring Harbor, N.Y., 1989.

1. A method of treating a subject having a cancer that exhibits a mutation in ARID1A and/or overexpresses CARM1 as compared to a similar non-cancerous cell comprising administering to said subject an inhibitor of IRE-1/XBP-1.

2. The method of claim 1, wherein the inhibitor is an IRE-1 selective inhibitor, an XBP-1 selective inhibitor, or an inhibitor of both IRE-1 and XBP-1.

3. The method of claim 1, further comprising treating said subject with a second cancer therapy, such as chemotherapy, radiotherapy, immunotherapy (e.g., checkpoint inhibitor), hormonal therapy, toxin therapy or surgery, either sequential or at the same time as said IRE-1/XBP-1 inhibitor.

4. The method of claim 3, wherein the immunotherapy is a checkpoint inhibitor therapy.

5. The method of claim 1, wherein the cancer overexpresses CARM1 as compared to a similar non-cancerous cell.

6. The method of claim 1, wherein the cancer exhibits a mutation in ARID1A.

7. The method of claim 1, wherein the cancer is an ovarian clear cell carcinoma cell or a high grade serous ovarian carcinoma cancer cell.

8. The method of claim 1, further comprising determining, prior to treating, that said subject carries an ARID1A-mutated cancer cell and/or a CARM1 overexpressing cancer cell.

9. The method of claim 8, wherein determining comprises:
(a) obtaining a sample from said subject that contains protein and/or nucleic acids; and
(b1) determining mutation status of an ARID1A protein or nucleic acid encoding ARID1A in said sample, or
(b2) determining the expression level of CARM1 in said sample.

10. The method of claim 9, wherein determining comprises a nucleic acid-based assay or a protein-based assay.

11. The method of claim 9, wherein said sample is a fluid sample.

12. The method of claim 11, wherein said fluid sample is blood, serum plasma, sputum, saliva, urine or nipple aspirate.

13. The method of claim 8, wherein said sample is a tissue sample.

14. The method of claim 13, wherein said tissue sample is a cancer tissue sample, such as a tumor biopsy.

15. The method of claim 1, wherein said subject is a human subject, such as a pediatric human subject.

16. The method of claim 1, wherein said subject is a non-human primate.

17. The method of claim 1, wherein said subject has previously been diagnosed with cancer, such as an OCCC cancer cell or an HGSOC cancer.

18. The method of claim 1, wherein said cancer is recurrent, primary, metastatic or multi-drug resistant.

19. The method of claim 1, wherein said IRE-1/XBP-1 inhibitor is administered more than once, such as daily, every other day, weekly, monthly and/or on a chronic basis.

20. The method of claim 1, wherein said method comprises administering both and IRE-1 inhibitor and an EZH2 inhibitor, either sequentially or at the same time.

21. A method of determining whether a subject having cancer should be treated with an inhibitor of IRE-1/XBP-1, wherein determining comprises:
(a) determining mutation status of an ARID1A protein or nucleic acid encoding ARID1A in a sample from said subject, and/or
(b) determining the expression level of CARM1 in a sample from said subject.

22. The method of claim 21, wherein the cancer is an ovarian clear cell carcinoma cell or a high grade serous ovarian carcinoma cancer cell, and/or is said cancer is recurrent, primary, metastatic or multi-drug resistant.

23. The method of claim 21, wherein determining comprises a nucleic acid-based assay or a protein-based assay.

24. The method of claim 21, wherein said sample is a fluid sample.

25. The method of claim 24, wherein said fluid sample is blood, serum plasma, sputum, saliva, urine or nipple aspirate.

26. The method of claim 21, wherein said sample is a tissue sample.

27. The method of claim 26, wherein said tissue sample is a cancer tissue sample, such as a tumor biopsy.

28. The method of claim 21, wherein said subject is a human subject, such as a pediatric human subject.

29. The method of claim 21, wherein said subject is a non-human primate.

30. The method of claim 21, wherein said subject has previously been diagnosed with cancer, such as an OCCC cancer cell or an HGSOC cancer.
